(19) United States
(12) Patent Application Publication
(10) Pub. No.: US 2011/0064803 A1
(43) Pub. Date: Mar. 17, 2011

(54) NANOPARTICULATE AND CONTROLLED RELEASE COMPOSITIONS COMPRISING VITAMIN K2

(75) Inventors: John G. Devane, Barrymore Athlone (IE); Paul Stark, Athlone (IE); Niall M.N. Fanning, Coosan Athlone (IE); Gurvinder Singh Rekhi, Suwanee, GA (US); Gary Liversidge, Westchester, PA (US); Scott Jenkins, Downington, PA (US)

(73) Assignee: ELAN PHARMA INTERNATIONAL LIMITED., COUNTY WESTMEATH (IE)

(21) Appl. No.: 11/569,206

(22) PCT Filed: May 9, 2006
(86) PCT No.: PCT/US06/17999

§ 371 Date: May 18, 2009

Related U.S. Application Data
(60) Provisional application No. 60/679,399, filed on May 10, 2005.

(30) Foreign Application Priority Data
Mar. 10, 2006 (US) 11-372857

(51) Int. Cl. A61K 9/64; A61K 9/14; A61K 31/122; A61K 9/20; A61K 9/22; A61K 9/48; A61K 9/54; A61K 9/50; A61P 19/10

(52) U.S. Cl. 424/456; 424/489; 514/682; 424/464; 424/468; 424/451; 424/458; 424/497

(57) ABSTRACT

The present invention is directed to compositions comprising a nanoparticulate vitamin K2 having improved bioavailability. The nanoparticulate vitamin K2 particles of the composition have an effective average particle size of less than about 2000 nm and are useful in the prevention and treatment of osteoporosis. The invention also relates to a controlled release composition comprising a vitamin K2 or a nanoparticulate vitamin K2 that in operation delivers the drug in a pulsed or multi-modal manner for the prevention and treatment of osteoporosis.

### FIELD OF INVENTION

The present invention relates to compositions and methods for the prevention and treatment of osteoporosis. In particular, the present invention relates to compositions comprising vitamin K2-containing particles and methods for making and using such a composition. In an embodiment of the invention, vitamin K2 is in nanoparticulate form. The present invention also relates to novel dosage forms for the controlled delivery of a Vitamin K2 composition.

### BACKGROUND OF INVENTION

### A. Background Regarding Vitamin K2

Vitamin K refers to a family of related compounds named for the first letter of the Danish word “koagulation”. As such, members of the vitamin K family are part of the clotting cascade which prevents uncontrolled bleeding in the event of cuts or internal broken blood vessels. Vitamin K2, also referred to as menantetrenone, is produced in the body by gastrointestinal bacteria. Unlike other vitamin K family members, vitamin K2 has the additional property of increasing bone mass and has proven therapeutic effects in patients suffering from osteoporosis.

Osteoporosis is a pathological state or disorder in which certain symptoms or risks occur due to a decrease in bone quantity that has reached a certain level. Loss of the balance between bone formation and bone resorption results in osteoporosis accompanied by a decrease in quantity of the bone. Vitamin K2 increases bone mass by three mechanisms: (1) accelerating osteogenesis; (2) inhibiting bone resorption; and (3) increasing serum levels of osteocalcin.

The chemical name for vitamin K2 is 2-methyl-3-[(2E,6E,10E)-3,7,11,15-tetramethylhexadeca-2,6,10,14-tetraen-1,4-napthoquinone. The molecular formula of vitamin K2 is C₃₁H₄₀O₂ and its molecular weight is 444.65. Vitamin K2 has the following structural formula:

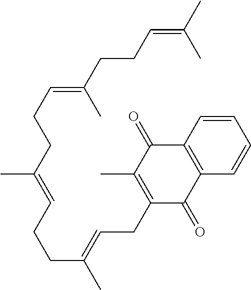

Vitamin K2 occurs as a yellow crystal, crystalline powder, waxy mass, or oily material. It is practically insoluble in water, slightly soluble in methanol, soluble in ethanol (99.5), and very soluble in hexane. It decomposes and the color becomes more intense by light.

Vitamin K2 may be administered as part of a dosage form offered under the registered trademark GLAKAY® by Eisai Co., Ltd. of Japan. GLAKAY® is administered to patients with ostoeoporosis for the improvement of bone mass decrease and pain relief. The usual dosage of GLAKAY® is 15 mg administered as an oral tablet three times daily after meals. Clinical studies have demonstrated that the absorption of GLAKAY® is greater according to the fat content of a meal, consistent with the fact that vitamin K2 is a fat soluble vitamin. Additionally, absorption of vitamin K2 increases when administered with fat because the presence of food delays gastric emptying allowing more time for vitamin K2 to dissolve. Thus, not only must GLAKAY® be taken after meals, but the meal should be high in fat content.

Vitamin K has high therapeutic value for the treatment of patients suffering from osteoporosis. However, given the need to take vitamin K2 three times a day and the further need to take vitamin K2 after high fat content meals, strict patient compliance is a critical factor in the efficacy of vitamin K2 in the treatment of osteoporosis. Moreover, such frequent administration often requires the attention of health care workers and contributes to the high cost associated with treatments involving vitamin K2. Thus, there is a need in the art for vitamin K2 compositions which overcome these and other problems associated with their use in the treatment of osteoporosis.

### B. Background Regarding Nanoparticulate Compositions

Nanoparticulate active agent compositions, first described in U.S. Pat. No. 5,145,684 (“the '684 patent”), are particles consisting of a poorly soluble therapeutic or diagnostic agent having adsorbed onto the surface thereof a non-crosslinked surface stabilizer. The '684 patent does not describe nanoparticulate compositions of vitamin K2.

Methods of making nanoparticulate active agent compositions are described in, for example, U.S. Pat. Nos. 5,518,187 and 5,862,999, both for “Method of Grinding Pharmaceutical Substances;” U.S. Pat. No. 5,718,388, for “Continuous Method of Grinding Pharmaceutical Substances;” and U.S. Pat. No. 5,510,118 for “Process of Preparing Therapeutic Compositions Containing Nanoparticles.”

Nanoparticulate active agent compositions are also described, for example, in U.S. Pat. No. 5,298,262 for “Use of Ionic Cloud Point Modifiers to Prevent Particle Aggregation During Sterilization;” U.S. Pat. No. 5,302,401 for “Method to Reduce Particle Size Growth During Lyophilization;” U.S. Pat. No. 5,318,767 for “X-Ray Contrast Compositions Useful in Medical Imaging;” U.S. Pat. No. 5,326,552 for “Novel Formulation For Nanoparticulate X-Ray Blood Pool Contrast Agents Using High Molecular Weight Non-ionic Surfactants;” U.S. Pat. No. 5,328,404 for “Method of X-Ray Imaging Using Iodinated Aromatic Propanedioates;” U.S. Pat. No. 5,336,507 for “Use of Charged Phospholipids to Reduce Nanoparticle Aggregation;” U.S. Pat. No. 5,340,564 for “Formulations Comprising Olin 10-G to Prevent Particle Aggregation and Increase Stability;” U.S. Pat. No. 5,346,702 for “Use of Non-Ionic Cloud Point Modifiers to Minimize Nanoparticulate Aggregation During Sterilization;” U.S. Pat. No. 5,349,957 for “Preparation and Magnetic Properties of Very Small Magnetic-Dextran Particles;” U.S. Pat. No. 5,352,459 for “Use of Purified Surface Modifiers to Prevent Particle Aggregation During Sterilization;” U.S. Pat. No. 5,399,363 and U.S. Pat. No. 5,494,683, both for “Surface Modified Anticancer Nanoparticles;” U.S. Pat. No. 5,401,492 for “Water Insoluble Non-Magnetic Manganese Particles as Magnetic Resonance Enhancement Agents;” U.S. Pat. No. 5,429,824 for “Use of Tyloxapol as a Nanoparticulate Stabilizer;” U.S. Pat. No. 5,447,710 for “Method for Making Nanoparticulate X-Ray Blood Pool Contrast Agents Using High Molecular Weight Non-ionic Surfactants;” U.S. Pat. No. 5,451,393 for “X-Ray Contrast Compositions Useful in Medical Imaging;” U.S. Pat. No. 5,466,440 for “Formulations of Oral Gastrointestinal Diagnostic X-Ray Contrast Agents in Combination with Pharmaceutically Acceptable Clays;” U.S. Pat. No. 5,470,583 for “Method of Preparing Nanoparticle Compositions Containing Charged Phospholipids to Reduce Aggregation;” U.S. Pat. No. 5,472,683 for “Nanoparticulate Diagnostic Mixed Carbamic Anhydrides as X-Ray Contrast Agents for Blood Pool and Lymphatic System Imaging;” U.S. Pat. No. 5,500,204 for “Nanoparticulate Diagnostic Dimers as X-Ray Contrast Agents for Blood Pool and Lymphatic System Imaging;” U.S. Pat. No. 5,518,738 for “Nanoparticulate NSAID Formulations;” U.S. Pat. No. 5,521,218 for “Nanoparticulate Iododipamide Derivatives for Use as X-Ray Contrast Agents;” U.S. Pat. No. 5,525,328 for “Nanoparticulate Diagnostic Diatrizoxy Ester X-Ray Contrast Agents for Blood Pool and Lymphatic System Imaging;” U.S. Pat. No. 5,543,133 for “Process of Preparing X-Ray Contrast Compositions Containing Nanoparticles;” U.S. Pat. No. 5,552,160 for “Surface Modified NSAID Nanoparticles;” U.S. Pat. No. 5,560,931 for “Formulations of Compounds as Nanoparticulate Dispersions in Digestible Oils or Fatty Acids;” U.S. Pat. No. 5,565,188 for “Polyalkylene Block Copolymers as Surface Modifiers for Nanoparticles;” U.S. Pat. No. 5,569,448 for “Sulfated Non-ionic Block Copolymer Surfactant as Stabilizer Coatings for Nanoparticle Compositions;” U.S. Pat. No. 5,571,536 for “Formulations of Compounds as Nanoparticulate Dispersions in Digestible Oils or Fatty Acids;” U.S. Pat. No. 5,573,749 for “Nanoparticulate Diagnostic Mixed Carboxylic Anydrides as X-Ray Contrast Agents for Blood Pool and Lymphatic System Imaging;” U.S. Pat. No. 5,573,750 for “Diagnostic Imaging X-Ray Contrast Agents;” U.S. Pat. No. 5,573,783 for “Redispersible Nanoparticulate Film Matrices With Protective Overcoats;” U.S. Pat. No. 5,580,579 for “Site-specific Adhesion Within the GI Tract Using Nanoparticles Stabilized by High Molecular Weight, Linear Poly(ethylene Oxide) Polymers;” U.S. Pat. No. 5,585,108 for “Formulations of Oral Gastrointestinal Therapeutic Agents in Combination with Pharmaceutically Acceptable Clays;” U.S. Pat. No. 5,587,143 for “Butylene Oxide-Ethylene Oxide Block Copolymers Surfactants as Stabilizer Coatings for Nanoparticulate Compositions;” U.S. Pat. No. 5,591,456 for “Milled Naproxen with Hydroxypropyl Cellulose as Dispersion Stabilizer;” U.S. Pat. No. 5,593,657 for “Novel Barium Salt Formulations Stabilized by Non-ionic and Anionic Stabilizers;” U.S. Pat. No. 5,622,938 for “Sugar Based Surfactant for Nanocrystals;” U.S. Pat. No. 5,628,981 for “Improved Formulations of Oral Gastrointestinal Diagnostic X-Ray Contrast Agents and Oral Gastrointestinal Therapeutic Agents;” U.S. Pat. No. 5,643,552 for “Nanoparticulate Diagnostic Mixed Carbonic Anhydrides as X-Ray Contrast Agents for Blood Pool and Lymphatic System Imaging;” U.S. Pat. No. 5,718,388 for “Continuous Method of Grinding Pharmaceutical Substances;” U.S. Pat. No. 5,718,919 for “Nanoparticles Containing the R(−)Enantiomer of Ibuprofen;” U.S. Pat. No. 5,747,001 for “Aerosols Containing Beclomethasone Nanoparticle Dispersions;” U.S. Pat. No. 5,834,025 for “Reduction of Intravenously Administered Nanoparticulate Formulation Induced Adverse Physiological Reactions;” U.S. Pat. No. 6,045,829 “Nanocrystalline Formulations of Human Immunodeficiency Virus (HIV) Protease Inhibitors Using Cellulosic Surface Stabilizers;” U.S. Pat. No. 6,068,858 for “Methods of Making Nanocrystalline Formulations of Human Immunodeficiency Virus (HIV) Protease Inhibitors Using Cellulosic Surface Stabilizers;” U.S. Pat. No. 6,153,225 for “Injectable Formulations of Nanoparticulate Naproxen;” U.S. Pat. No. 6,165,506 for “New Solid Dose Form of Nanoparticulate Naproxen;” U.S. Pat. No. 6,221,400 for “Methods of Treating Mammals Using Nanocrystalline Formulations of Human Immunodeficiency Virus (HIV) Protease Inhibitors;” U.S. Pat. No. 6,264,922 for “Nebulized Aerosols Containing Nanoparticle Dispersions;” U.S. Pat. No. 6,267,989 for “Methods for Preventing Crystal Growth and Particle Aggregation in Nanoparticle Compositions;” U.S. Pat. No. 6,270,806 for “Use of PEG-Derivatized Lipids as Surface Stabilizers for Nanoparticulate Compositions;” U.S. Pat. No. 6,316,029 for “Rapidly Disintegrating Solid Oral Dosage Form,” U.S. Pat. No. 6,375,986 for “Solid Dose Nanoparticulate Compositions Comprising a Synergistic Combination of a Polymeric Surface Stabilizer and Dioctyl Sodium Sulfosuccinate;” U.S. Pat. No. 6,428,814 for “Bioadhesive Nanoparticulate Compositions Having Cationic Surface Stabilizers;” U.S. Pat. No. 6,431,478 for “Small Scale Mill;” and U.S. Pat. No. 6,432,381 for “Methods for Targeting Drug Delivery to the Upper and/or Lower Gastrointestinal Tract,” U.S. Pat. No. 6,582,285 for “Apparatus for Sanitary Wet Milling;” and U.S. Pat. No. 6,592,903 for “Nanoparticulate Dispersions Comprising a Synergistic Combination of a Polymeric Surface Stabilizer and Dioctyl Sodium Sulfosuccinate;” U.S. Pat. No. 6,656,504 for “Nanoparticulate Compositions Comprising Amorphous Cyclosporine;” U.S. Pat. No. 6,742,734 for “System and Method for Milling Materials;” U.S. Pat. No. 6,745,962 for “Small Scale Mill and Method Thereof;” U.S. Pat. No. 6,811,767 for “Liquid droplet aerosols of nanoparticulate drugs;” U.S. Pat. No. 6,908,626 for “Compositions having a combination of immediate release and controlled release characteristics;” U.S. Pat. No. 6,969,529 for “Nanoparticulate compositions comprising copolymers of vinyl pyrrolidone and vinyl acetate as surface stabilizers;” U.S. Pat. No. 6,976,647 for “System and Method for Milling Materials;” and U.S. Pat. No. 6,991,191 for “Method of Using a Small Scale Mill;” all of which are specifically incorporated by reference. In addition, U.S. Patent Publication No. 20020012675 A1, for “Controlled Release Nanoparticulate Compositions;” U.S. Patent Publication No. 20050276974 for “Nanoparticulate Fibrate Formulations;” U.S. Patent Publication No. 20050238725 for “Nanoparticulate compositions having a peptide as a surface stabilizer,” U.S. Patent Publication No. 20050233001 for “Nanoparticulate megestrol formulations;” U.S. Patent Publication No. 20050147664 for “Compositions comprising antibodies and methods of using the same for targeting nanoparticulate active agent delivery;” U.S. Patent Publication No. 20050063913 for “Novel metaxalone compositions;” U.S. Patent Publication No. 20050042177 for “Novel compositions of sildenafil free base;” U.S. Patent Publication No. 20050031691 for “Gel stabilized nanoparticulate active agent compositions;” U.S. Patent Publication No. 20050019412 for “Novel glipizide compositions;” U.S. Patent Publication No. 20050004049 for “Novel griseofulvin compositions;” U.S. Patent Publication No. 20040258758 for “Nanoparticulate topiramate formulations;” U.S. Patent Publication No. 20040258757 for “Liquid dosage compositions of stable nanoparticulate active agents;” U.S. Patent Publication No. 20040229038 for “Nanoparticulate meloxicam formulations;” U.S. Patent Publication No. 20040208833 for “Novel fluticasone formulations;” U.S. Patent Publication No. 20040195413 for “Compositions and method for milling materials;” U.S. Patent Publication No. 20040156895 for “Solid dosage forms comprising pullulan;” U.S. Patent Publication No. U.S. Patent Publication No. U.S. Patent Publication No. 20040156872 for “Novel nimesulide compositions;” U.S. Patent Publication No. 20040141925 for “Novel triamcinolone compositions;” U.S. Patent Publication No. 20040115134 for “Novel nifedipine compositions;” U.S. Patent Publication No. 20040105889 for “Low viscosity liquid dosage forms;” U.S. Patent Publication No. 20040105778 for “Gamma irradiation of solid nanoparticulate active agents;” U.S. Patent Publication No. 20040101566 for “Novel benzoyl peroxide compositions;” U.S. Patent Publication No. 20040057905 for “Nanoparticulate beclomethasone dipropionate compositions;” U.S. Patent Publication No. 20040033267 for “Nanoparticulate compositions of angiogenesis inhibitors;” U.S. Patent Publication No. 20040033202 for “Nanoparticulate sterol formulations and novel sterol combinations;” U.S. Patent Publication No. 20040018242 for “Nanoparticulate nystatin formulations;” U.S. Patent Publication No. 20040015134 for “Drug delivery systems and methods;” U.S. Patent Publication No. 20030232796 for “Nanoparticulate polycosanol formulations & novel polycosanol combinations;” U.S. Patent Publication No. 20030215502 for “Fast dissolving dosage forms having reduced friability;” U.S. Patent Publication No. 20030185869 for “Nanoparticulate compositions having lysozyme as a surface stabilizer;” U.S. Patent Publication No. 20030181411 for “Nanoparticulate compositions of mitogen-activated protein (MAP) kinase inhibitors;” U.S. Patent Publication No. 20030137067 for “Compositions having a combination of immediate release and controlled release characteristics;” U.S. Patent Publication No. 20030108616 for “Nanoparticulate compositions comprising copolymers of vinyl pyrrolidone and vinyl acetate as surface stabilizers;” U.S. Patent Publication No. 20030095928 for “Nanoparticulate insulin;” U.S. Patent Publication No. 20030087308 for “Method for high through put screening using a small scale mill or microfluidics;” U.S. Patent Publication No. 20030023203 for “Drug delivery systems & methods;” U.S. Patent Publication No. 20020179758 for “System and method for milling materials; and U.S. Patent Publication No. 20010053664 for “Apparatus for sanitary wet milling,” describe nanoparticulate active agent compositions and are specifically incorporated by reference. None of these references describe compositions of nanoparticulate vitamin K2.

Amorphous small particle compositions are described, for example, in U.S. Pat. Nos. 4,783,484 for “Particulate Composition and Use Thereof as Antimicrobial Agent;” 4,826,689 for “Method for Making Uniformly Sized Particles from Water-Insoluble Organic Compounds;” U.S. Pat. No. 4,997,454 for “Method for Making Uniformly-Sized Particles From Insoluble Compounds;” U.S. Pat. No. 5,741,522 for “Ultrasmall, Non-aggregated Porous Particles of Uniform Size for Entrapping Gas Bubbles Within and Methods;” and 5,776,496, for “Ultrasmall Porous Particles for Enhancing Ultrasound Back Scatter.”

Because vitamin K2 is practically insoluble in water, significant bioavailability can be problematic. Moreover, vitamin K2 must be taken three times a day with high fat content meals. Thus, there is a need in the art for nanoparticulate vitamin K2 formulations which overcome these and other problems associated with the use of vitamin K2 in the prevention and treatment of osteoporosis. The present invention satisfies this need.

The present invention then relates in part to a nanoparticulate vitamin K2 composition for the treatment of osteoporosis. The nanoparticulate vitamin K2 compositions of the invention have enhanced bioavailability, allowing a smaller dose to give the same in vivo blood levels. The compositions of the invention also have enhanced bioavailability in the fasting state that would match that seen in the fed state and thereby eliminate the requirement to take vitamin K2 with food. Additionally, the present invention relates to controlled release compositions of vitamin K2 which eliminate the need for frequent administration.

### C. Background Regarding Controlled Release Vitamin K2 Compositions

The objective of many controlled release drug formulations is to produce a substantially constant release of the drug compound. Indeed, it is often a specific object of these formulations to minimize the variation in plasma concentration levels associated with conventional frequent dosage regimes. Another objective of controlled release drug formulations is to hasten the onset of action by minimizing to time from the administration of the drug to the achievement of a therapeutically effective plasma concentration. A further objective of controlled release drug formulations is to maintain a therapeutically effective plasma concentration throughout the dosing interval. The achievement of two or all three of these objectives in conventional drug formulations, however, is problematic. Thus, controlled release compositions or formulations which combine the benefits of at least two different release profiles to achieve a resultant plasma profile that minimizes variations in plasma concentration levels and/or provides rapid onset of action and/or maintains a therapeutically effective plasma concentration throughout the dosing interval is desirable.

Conventional frequent dosage regimes in which an immediate release (IR) dosage form is administered at periodic intervals typically give rise to a pulsatile plasma profile. In such cases, a peak in the plasma drug concentration is observed after administration of each IR dose with periods of lower drug concentration observed between consecutive administration time points. Such dosage regimes and the resultant pulsatile plasma profiles associated therewith can have particular pharmacological and therapeutic effects that are beneficial for certain drug therapies. For example, the wash out period provided by the drop in the plasma concentration of the active ingredient between peaks has been thought to be a contributing factor in reducing or preventing patient tolerance to various types of drugs.

For certain drugs, some of the therapeutic and pharmacological effects intrinsic in a pulsatile system may be lost or diminished as a result of the constant or nearly constant plasma concentration levels achieved by continuous release drug delivery systems. Thus, modified release compositions or formulations which substantially mimic the release of frequent IR dosage regimes while reducing the need for frequent dosing is desirable.

A typical example of a drug which may produce tolerance in patients is methylphenidate. Methylphenidate, or α-phenyl-2-piperidine acetic acid methyl ester, is a stimulant affecting the central nervous and respiratory systems and is primarily used in the treatment of attention deficit hyperactivity disorder (ADHD). After absorption from the gastrointestinal tract (GIT), drug effects persist for 3-6 hours after oral administration of conventional IR tablets or up to about 8 hours after oral administration of extended release formulations. The total dosage is typically in the range of 5-30 mg per day, in exceptional cases rising to 60 mg/day. Under conventional dosage regimes, methylphenidate is given twice daily, typically with one dose given before breakfast and a second dose given before lunch. The last daily dose is preferably given several hours before retiring. Adverse effects associated with methylphenidate treatment include insomnia and the development of patient tolerance.

WO 98/14168 (Alza Corp.) teaches a dosage form and a method of administering methylphenidate in a sustained and constantly ascending rate. The dosage form disclosed comprises a plurality of beads comprising a hydrogel matrix with increasing amounts of the active ingredient therein, coated with varying amounts of a release rate controlling material. Appropriate combinations of the active ingredient dose and the number and thickness coating layers can be selected to give an ascending release profile in which the plasma concentration of the active ingredient continually increases over a given period of time. An object of WO 98/14168 is to release a dosage form at a constantly ascending rate specifically to avoid uneven blood levels (characterized by peaks and troughs) associated with conventional treatments using immediate release dosage formulations. As a result, this formulation does not deliver the active ingredient in either a pulsatile or a bimodal manner.

WO 97/03672 (Chiroscience Ltd.) discloses that methylphenidate exhibits a therapeutic effect when administered in the form of a racemic mixture or in the form of a single isomer (such as the RR d-threo enantiomer). Further, WO 97/03763 (Chiroscience Ltd.) discloses a sustained release formulation containing d-threo methylphenidate (dtmp). This disclosure teaches the use of a composition comprising a coating through which the dtmp passes in order to attain sustained release and achieve serum levels (of the active ingredient) of at least 50% cmax over a period of at least 8 hours. As above, this formulation does not deliver the active ingredient in either a pulsatile or a bimodal manner.

Shah et al., J Cont. Rel. (1989) 9:169-175 purports to disclose that certain types of hydroxypropyl methylcellulose ethers compressed into a solid dosage form with a therapeutic agent may produce a bimodal release profile. However, it is noted that while polymers from one supplier yielded a bimodal profile, the same polymers with almost identical product specifications obtained from a different source gave non-bimodal release profiles.

Giunchedi et al., Int. J. Pharm (1991) 77:177-181 discloses the use of a hydrophilic matrix multiple-unit formulation for the pulsed release of ketoprofen. Giunchedi et al. teach that ketoprofen is rapidly eliminated from the blood after dosing (plasma half-life 1-3 hours) and consecutive pulses of drug may be more beneficial than constant release for some treatments. The multiple-unit formulation disclosed comprises four identical hydrophilic matrix tablets placed in a gelatin capsule. Although the in vivo studies show two peaks in the plasma profile there is no well defined wash out period and the variation between the peak and trough plasma levels is small.

Conte et al., Drug Dev. Ind. Pharm, (1989) 15:2583-2596 and EP 0 274 734 (Phamidea Srl) teach the use of a three layer tablet for delivery of ibuprofen in consecutive pulses. The three layer tablet is made up of a first layer containing the active ingredient, a barrier layer (the second layer) of semi-permeable material which is interposed between the first layer and a third layer containing an additional amount of active ingredient. The barrier layer and the third layer are housed in an impermeable casing. The first layer dissolves upon contact with a dissolving fluid while the third layer is only available after dissolution or rupture of the barrier layer. In such a tablet the first portion of active ingredient must be released instantly. This approach also requires the provision of a semi-permeable layer between the first and third layers in order to control the relative rates of delivery of the two portions of active ingredient. Additionally, rupture of the semi-permeable layer leads to uncontrolled dumping of the second portion of the active ingredient which may not be desirable.

U.S. Pat. No. 5,158,777 (E. R. Squibb & Sons Inc.) discloses a formulation comprising captopril within an enteric or delayed release coated pH stable core combined with additional captopril which is available for immediate release following administration. In order to form the pH stable core, chelating agents such as disodium edetate or surfactants such as polysorbate 80 are used either alone or in combination with a buffering agent. The compositions have an amount of captopril available for immediate release following oral administration and an additional amount of pH stabilized captopril available for release in the colon.

U.S. Pat. Nos. 4,728,512, 4,794,001 and 4,904,476 (American Home Products Corp.) relate to preparations providing three distinct releases. The preparation contains three groups of spheroids containing an active medicinal substance: the first group of spheroids is uncoated and rapidly disintegrates upon ingestion to release an initial dose of medicinal substance; the second group of spheroids is coated with a pH sensitive coat to provide a second dose; and the third group of spheroids is coated with a pH independent coat to provide to third dose. The preparation is designed to provide repeated release of medicinal substances which are extensively metabolized presystemically or have relatively short elimination half-lives.

U.S. Pat. No. 5,837,284 (Mehta et al) discloses a methylphenidate dosage form having immediate release and delayed release particles. The delayed release is provided by the use of ammonio methacrylate pH independent polymers combined with certain fillers.

### SUMMARY OF THE INVENTION

The present invention relates to nanoparticulate compositions comprising vitamin K2. The compositions comprise nanoparticulate vitamin K2 particles, and at least one surface stabilizer adsorbed on or associated with the surface of the vitamin K2 particles. The nanoparticulate vitamin K2 particles have an effective average particle size of less than about 2,000 mm.

The present invention provides nanoparticulate vitamin K2 compositions which overcome the poor bioavailability of conventional non-nanoparticulate vitamin K2 and eliminate the requirement to take the product with food. The present invention also provides controlled release compositions of vitamin K2 which eliminate the need to take vitamin K2 three times a day.

A preferred dosage form of the invention is a solid dosage form, although any pharmaceutically acceptable dosage form can be utilized.

Another aspect of the invention is directed to pharmaceutical compositions comprising a nanoparticulate vitamin K2 particle and at least one surface stabilizer, a pharmaceutically acceptable carrier, as well as any desired excipients.

One embodiment of the invention encompasses a nanoparticulate vitamin K2 composition, wherein the pharmacokinetic profile of the nanoparticulate vitamin K2 is not affected by the fed or fasted state of a subject ingesting the composition.

In yet another embodiment, the invention encompasses a nanoparticulate vitamin K2 composition, wherein administration of the composition to a subject in a fasted state is bioequivalent to administration of the composition to a subject in a fed state.

Another embodiment of the invention is directed to nanoparticulate vitamin K2 compositions comprising one or more additional compounds useful in the prevention and treatment of osteoporosis.

This invention further discloses a method of making the inventive nanoparticulate vitamin K2 composition. Such a method comprises contacting the nanoparticulate vitamin K2 with at least one surface stabilizer for a time and under conditions sufficient to provide a stabilized nanoparticulate vitamin K2 composition.

The present invention is also directed to methods of treatment including but not limited to, the prevention and treatment of osteoporosis using the novel nanoparticulate vitamin K2 compositions disclosed herein. Such methods comprise administering to a subject a therapeutically effective amount of a nanoparticulate vitamin K2. Other methods of treatment using the nanoparticulate compositions of the invention are known to those of skill in the art.

The present invention further relates to a controlled release composition comprising a vitamin K2 or a nanoparticulate vitamin K2 which, in operation, produces a plasma profile substantially similar to the plasma profile produced by the administration of two or more IR dosage forms given sequentially.

The present invention relates to a controlled release composition that, in operation, delivers a vitamin K2 or a nanoparticulate vitamin K2, in a pulsatile or continuous manner, preferably during a period of up to twenty-four hours.

Another object of the invention is to provide a controlled release composition which substantially mimics the pharmacological and therapeutic effects produced by the administration of three or more IR dosage forms comprising a vitamin K2 or a nanoparticulate vitamin K2 given sequentially.

Another object of the invention is to provide a controlled release composition which substantially reduces or eliminates the development of patient tolerance to a vitamin K2 or a nanoparticulate vitamin K2.

Another object of the invention is to provide a controlled release composition in which a first portion of the composition, i.e., a vitamin K2 or a nanoparticulate vitamin K2, is released immediately upon administration and a second portion of the active ingredient is released rapidly after an initial delay period and a third portion of the active ingredient is released rapidly after an initial delay period in a multi-modal manner

Another object of the invention is to formulate the dosage in the form of erodable formulations, diffusion controlled formulations, or osmotic controlled formulations.

Another object of the invention is to provide a controlled release composition capable of releasing a vitamin K2 or a nanoparticulate vitamin K2, in a bimodal or multi-modal manner in which a first portion of the active is released either immediately or after a delay time to provide a pulse of drug release and one or more additional portions of the vitamin K2 or a nanoparticulate vitamin K2 is released, after a respective lag time, to provide additional pulses of drug release during a period of up to twenty-four hours.

Another object of the invention is to provide solid oral dosage forms comprising a controlled release composition comprising a vitamin K2 or a nanoparticulate vitamin K2.

Yet another object of the invention is to provide a method for the treatment of pain and/or inflammation comprising the administration of a composition of the present invention.

Both the foregoing general description and the following detailed description are exemplary and explanatory and are intended to provide further explanation of the invention as claimed. Other objects, advantages, and novel features will be readily apparent to those skilled in the art from the following detailed description of the invention.

### DETAILED DESCRIPTION OF THE INVENTION

### I. Nanoparticulate Vitamin K2 Compositions

The present invention is directed to nanoparticulate compositions comprising a vitamin K2. The compositions comprise a vitamin K2 and preferably at least one surface stabilizer adsorbed on the surface of the drug. The vitamin K2 particles have an effective average particle size of less than about 2000 nm.

As taught by the '684 patent, and as exemplified in the examples below, not every combination of surface stabilizer and active agent will results in a stable nanoparticulate composition. It was surprisingly discovered that stable, nanoparticulate vitamin K2 formulations can be made.

Advantages of the nanoparticulate vitamin K2 formulation of the invention as compared to conventional, non-nanoparticulate microcrystalline vitamin K2 formulations include, but are not limited to: (1) smaller tablet or other solid dosage form size; (2) smaller doses of drug required to obtain the same pharmacological effect; (3) increased bioavailability; (4) substantially similar pharmacokinetic profiles of the vitamin K2 compositions when administered in the fed versus the fasted state; (5) bio equivalency of the vitamin K2 compositions when administered in the fed versus the fasted state; (6) an increased rate of dissolution for the vitamin K2 compositions; (7) the vitamin K2 compositions can be used in conjunction with other active agents useful in the prevention and treatment of osteoporosis.

The present invention also includes nanoparticulate vitamin K2 compositions together with one or more non-toxic physiologically acceptable carriers, adjuvants, or vehicles, collectively referred to as carriers. The compositions can be formulated for parental injection (e.g., intravenous, intramuscular, or subcutaneous), oral administration in solid, liquid, or aerosol form, vaginal, nasal, rectal, ocular, local (powders, ointments, or drops), buccal, intracisternal, intraperitoneal, or topical administrations, and the like.

A preferred dosage form of the invention is a solid dosage form, although any pharmaceutically acceptable dosage form can be utilized. Exemplary solid dosage forms include, but are not limited to, tablets, capsules, sachets, lozenges, powders, pills, or granules, and the solid dosage form can be, for example, a fast melt dosage form, controlled release dosage form, lyophilized dosage form, delayed release dosage form, extended release dosage form, pulsatile release dosage form, mixed immediate release and controlled release dosage form, or a combination thereof. A solid dose tablet formulation is preferred.

### A. Definitions

The present invention is described herein using several definitions, as set forth below and throughout the application.

As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein with reference to particles of vitamin K2, “stable” means that the vitamin K2 particles do not appreciably flocculate or agglomerate due to interparticle attractive forces or otherwise spontaneously increase in particle size.

The term “effective average particle size” as used herein means that at least 50% of the particles have a size, by weight or other suitable measurement (e.g., volume, number, etc.), of a certain amount when measured by, for example, sedimentation field flow fractionation, photon correlation spectroscopy, light scattering, disk centrifugation, and other techniques known to those of skill in the art. Accordingly, “effective average particle size of less than about 2000 nm” means that at least 50% of the vitamin K2 particles have a size, by weight or other suitable measurement (i.e., volume, number, etc.), of less than about 2000 nm,

The term “conventional” or “non-nanoparticulate” vitamin K2 means vitamin K2 which is solubilized or which has an effective average particle size of greater than about 2000 nm. Nanoparticulate active agents as defined herein have an effective average particle size of less than about 2000 nm.

As used herein, the phrase “therapeutically effective amount” shall mean the drug dosage that provides the specific pharmacological response for which the drug is administered in a significant number of subjects in need of such treatment. It is emphasized that a therapeutically effective amount of a drug that is administered to a particular subject in a particular instance will not always be effective in treating the conditions/diseases described herein, even though such dosage is deemed to be a therapeutically effective amount by those of skill in the art.

The term “particulate” as used herein refers to a state of matter which is characterized by the presence of discrete particles, pellets, beads or granules irrespective of their size, shape or morphology. The term “multiparticulate” as used herein means a plurality of discrete, or aggregated, particles, pellets, beads, granules or mixture thereof irrespective of their size, shape or morphology.

### B. Preferred Characteristics of the Nanoparticulate Vitamin K2 Compositions of the Invention

1. Increased Bioavailability

The nanoparticulate vitamin K2 formulations of the invention are proposed to exhibit increased bioavailability, and require smaller doses as compared to prior conventional vitamin K2 formulations.

2. Improved Pk Profiles

The invention also preferably provides compositions comprising nanoparticulate vitamin K2 and having a desirable pharmacokinetic profile when administered to mammalian subjects. The desirable pharmacokinetic profile of the compositions comprising vitamin K2 preferably includes, but is not limited to: (1) a Cₘₐₓ for vitamin K2, when assayed in the plasma of a mammalian subject following administration, that is preferably greater than the C for a non-nanoparticulate formulation of vitamin K2, administered at the same dosage; and/or (2) an AUC for vitamin K2, when assayed in the plasma of a mammalian subject following administration, that is preferably greater than the AUC for a non-nanoparticulate formulation of vitamin K2, administered at the same dosage; and/or (3) a Tₘₐₓ for vitamin K2, when assayed in the plasma of a mammalian subject following administration, that is preferably less than the Tₘₐₓ for a non-nanoparticulate formulation of vitamin K2, administered at the same dosage. The desirable pharmacokinetic profile, as used herein, is the pharmacokinetic profile measured after the initial dose of vitamin K2.

In one embodiment, a composition comprising nanoparticulate vitamin K2 exhibits in comparative pharmacokinetic testing with a non-nanoparticulate formulation of vitamin K2, administered at the same dosage, a Tₘₐₓ not greater than about 90%, not greater than about 80%, not greater than about 70%, not greater than about 60%, not greater than about 50%, not greater than about 30%, not greater than about 25%, not greater than about 20%, not greater than about 15%, not greater than about 10%, or not greater than about 5% of the Tₘₐₓ exhibited by the non-nanoparticulate vitamin IC₂ formulation.

In another embodiment, the composition comprising nanoparticulate vitamin K2 exhibits in comparative pharmacokinetic testing with a non-nanoparticulate formulation of vitamin K2, administered at the same dosage, a Cₘₐₓ which is at least about 50%, at least about 100%, at least about 200%, at least about 300%, at least about 400%, at least about 500%, at least about 600%, at least about 700%, at least about 800%, at least about 900%, at least about 1000%, at least about 1100%, at least about 1200%, at least about 1300%, at least about 1400%, at least about 1500%, at least about 1600%, at least about 1700%, at least about 1800%, or at least about 1900% greater than the Cₘₐₓ exhibited by the non-nanoparticulate vitamin K2 formulation.

In yet another embodiment, the composition comprising nanoparticulate vitamin K2 exhibits in comparative pharmacokinetic testing with a non-nanoparticulate formulation of vitamin K2, administered at the same dosage, an AUC which is at least about 25%, at least about 50%, at least about 75%, at least about 100%, at least about 125%, at least about 150%, at least about 175%, at least about 200%, at least about 225%, at least about 250%, at least about 275%, at least about 300%, at least about 350%, at least about 400%, at least about 450%, at least about 500%, at least about 550%, at least about 600%, at least about 750%, at least about 700%, at least about 750%, at least about 800%, at least about 850%, at least about 900%, at least about 950%, at least about 1000%, at least about 1050%, at least about 1100%, at least about 1150%, or at least about 1200% greater than the AUC exhibited by the non-nanoparticulate vitamin K2 formulation.

3. The Pharmacokinetic Profiles of the Vitamin K2 Compositions of the Invention are not Affected by the Fed or Fasted State of the Subject Ingesting the Compositions

The invention encompasses vitamin K2 composition wherein the pharmacokinetic profile of vitamin K2 is not substantially affected by the fed or fasted state of a subject ingesting the composition. This means that there is no substantial difference in the quantity of drug absorbed or the rate of drug absorption when the nanoparticulate vitamin K2 compositions are administered in the fed versus the fasted state.

For conventional vitamin K2 formulations, i.e., GLAKAY®, the absorption of vitamin K2 is increased when administered with food. This difference in absorption observed with conventional vitamin K2 formulations is undesirable. The vitamin K2 formulations of the invention overcome this problem, as the vitamin K2 formulations reduce or preferably substantially eliminate significantly different absorption levels when administered under fed as compared to fasting conditions.

Benefits of a dosage form which substantially eliminates the effect of food include an increase in subject convenience, thereby increasing subject compliance, as the subject does not need to ensure that they are taking a dose either with or without food. This is significant, as with poor subject compliance an increase in the medical condition for which the drug is being prescribed may be observed, i.e., osteoporosis for poor subject compliance with vitamin K2.

4. Bioequivalency of Vitamin K2 Compositions of the Invention When Administered in the Fed Versus the Fasted State

The invention also encompasses provides a nanoparticulate vitamin K2 composition in which administration of the composition to a subject in a fasted state is bioequivalent to administration of the composition to a subject in a fed state.

The difference in absorption of the vitamin K2 compositions of the invention, when administered in the fed versus the fasted state, preferably is less than about 35%, less than about 30%, less than about 25%, less than about 20%, less than about 15%, less than about 10%, less than about 5%, or less than about 3%.

In one embodiment of the invention, the invention encompasses compositions comprising nanoparticulate vitamin K2, wherein administration of the composition to a subject in a fasted state is bioequivalent to administration of the composition to a subject in a fed state, in particular as defined by Cₘₐₓ and AUC guidelines given by the U.S. Food and Drug Administration and the corresponding European regulatory agency (EMEA). Under U.S. FDA guidelines, two products or methods are bioequivalent if the 90% Confidence Intervals (CI) for AUC and Cₘₐₓ are between 0.80 to 1.25 (Tₘₐₓ measurements are not relevant to bioequivalence for regulatory purposes). To show bioequivalency between two compounds or administration conditions pursuant to Europe's EMEA guidelines, the 90% CI for AUC must be between 0.80 to 1.25 and the 90% CI for Cₘₐₓ must between 0.70 to 1.43.

5. Dissolution Profiles of the Vitamin K2 Compositions of the Invention

The nanoparticulate vitamin K2 compositions of the invention are proposed to have unexpectedly dramatic dissolution profiles. Rapid dissolution of an administered active agent is preferable, as faster dissolution generally leads to faster onset of action and greater bioavailability. To improve the dissolution profile and bioavailability of the vitamin K2, it would be useful to increase the drug's dissolution so that it could attain a level close to 100%.

The vitamin K2 compositions of the invention preferably have a dissolution profile in which within about 5 minutes at least about 20% of the composition is dissolved. In other embodiments of the invention, at least about 30% or at least about 40% of vitamin K2 composition is dissolved within about 5 minutes. In yet other embodiments of the invention, preferably at least about 40%, at least about 50%, at least about 60%, at least about 70%, or at least about 80% of the vitamin K2 composition is dissolved within about 10 minutes. Finally, in another embodiment of the invention, preferably at least about 70%, at least about 80%, at least about 90%, or at least about 100% of the vitamin K2 composition is dissolved within about 20 minutes.

Dissolution is preferably measured in a medium which is discriminating. Such a dissolution medium will produce two very different dissolution curves for two products having very different dissolution profiles in gastric juices; i.e., the dissolution medium is predictive of in vivo dissolution of a composition. An exemplary dissolution medium is an aqueous medium containing the surfactant sodium lauryl sulfate at 0.025 M. Determination of the amount dissolved can be carried out by spectrophotometry. The rotating blade method (European Pharmacopoeia) can be used to measure dissolution.

6. Redispersibility Profiles of the Vitamin K2 Compositions of the Invention

An additional feature of the vitamin K2 compositions of the invention is that the compositions redisperse such that the effective average particle size of the redispersed vitamin K2 particles is less than about 2 microns. This is significant, as if upon administration the vitamin K2 compositions of the invention did not redisperse to a substantially nanoparticulate particle size, then the dosage form may lose the benefits afforded by formulating the vitamin K2 into a nanoparticulate particle size.

This is because nanoparticulate active agent compositions benefit from the small particle size of the active agent; if the active agent does not redisperse into the small particle sizes upon administration, then “clumps” or agglomerated active agent particles are formed, owing to the extremely high surface free energy of the nanoparticulate system and the thermodynamic driving force to achieve an overall reduction in free energy. With the formation of such agglomerated particles, the bioavailability of the dosage form may fall well below that observed with the liquid dispersion form of the nanoparticulate active agent.

Moreover, the nanoparticulate vitamin K2 compositions of the invention exhibit dramatic redispersion of the nanoparticulate vitamin K2 particles upon administration to a mammal, such as a human or animal, as demonstrated by reconstitution/redispersion in a biorelevant aqueous media such that the effective average particle size of the redispersed vitamin K2 particles is less than about 2 microns. Such biorelevant aqueous media can be any aqueous media that exhibit the desired ionic strength and pH, which form the basis for the biorelevance of the media. The desired pH and ionic strength are those that are representative of physiological conditions found in the human body. Such biorelevant aqueous media can be, for example, aqueous electrolyte solutions or aqueous solutions of any salt, acid, or base, or a combination thereof, which exhibit the desired pH and ionic strength. Such redispersion in a biorelevant media is predictive of in vivo efficacy of the vitamin K2 dosage form.

Biorelevant pH is well known in the art. For example, in the stomach, the pH ranges from slightly less than 2 (but typically greater than 1) up to 4 or 5. In the small intestine the pH can range from 4 to 6, and in the colon it can range from 6 to 8. Biorelevant ionic strength is also well known in the art. Fasted state gastric fluid has an ionic strength of about 0.1M while fasted state intestinal fluid has an ionic strength of about 0.14. See e.g., Lindahl et al., “Characterization of Fluids from the Stomach and Proximal Jejunum in Men and Women,” Pharm. Res., 14 (4): 497-502 (1997).

It is believed that the pH and ionic strength of the test solution is more critical than the specific chemical content. Accordingly, appropriate pH and ionic strength values can be obtained through numerous combinations of strong acids, strong bases, salts, single or multiple conjugate acid-base pairs (i.e., weak acids and corresponding salts of that acid), monoprotic and polyprotic electrolytes, etc.

Representative electrolyte solutions can be, but are not limited to, HCl solutions, ranging in concentration from about 0.001 to about 0.1 N, and NaCl solutions, ranging in concentration from about 0.001 to about 0.1 M, and mixtures thereof. For example, electrolyte solutions can be, but are not limited to, about 0.1 N HCl or less, about 0.01 N HCl or less, about 0.001 N HCl or less, about 0.1 M NaCl or less, about 0.01 M NaCl or less, about 0.001 M NaCl or less, and mixtures thereof. Of these electrolyte solutions, 0.01 N HCl and/or 0.1 M NaCl, are most representative of fasted human physiological conditions, owing to the pH and ionic strength conditions of the proximal gastrointestinal tract.

Electrolyte concentrations of 0.001 N HCl, 0.01 N HCl, and 0.1 N HCl correspond to pH 3, pH 2, and pH 1, respectively. Thus, a 0.01 N HCl solution simulates typical acidic conditions found in the stomach. A solution of 0.1 M NaCl provides a reasonable approximation of the ionic strength conditions found throughout the body, including the gastrointestinal fluids, although concentrations higher than 0.1 M may be employed to simulate fed conditions within the human GI tract.

Exemplary solutions of salts, acids, bases or combinations thereof, which exhibit the desired pH and ionic strength, include but are not limited to phosphoric acid/phosphate salts+sodium, potassium and calcium salts of chloride, acetic acid/acetate salts+sodium, potassium and calcium salts of chloride, carbonic acid/bicarbonate salts+sodium, potassium and calcium salts of chloride, and citric acid/citrate salts+sodium, potassium and calcium salts of chloride.

In other embodiments of the invention, the redispersed vitamin K2 particles of the invention (redispersed in an aqueous, biorelevant, or any other suitable media) have an effective average particle size of less than about 2000 nm, less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods. Such methods suitable for measuring effective average particle size are known to a person of ordinary skill in the art.

Redispersibility can be tested using any suitable means known in the art. See e.g., the example sections of U.S. Pat. No. 6,375,986 for “Solid Dose Nanoparticulate Compositions Comprising a Synergistic Combination of a Polymeric Surface Stabilizer and Dioctyl Sodium Sulfosuccinate.”

7. Vitamin K2 Compositions Used in Conjunction with Other Active Agents

The vitamin K2 compositions of the invention can additionally comprise one or more compounds useful in treating osteoporosis, or the vitamin K2 compositions can be administered in conjunction with such a compound. Examples of such compounds include, but are not limited to, risedronic acid, estrogens, calcitonins, and bisphoshonates.

### C. Nanoparticulate Vitamin K2 Compositions

The invention provides compositions comprising vitamin K2 particles and at least one surface stabilizer. The surface stabilizers preferably are adsorbed on, or associated with, the surface of the vitamin K2 particles. Surface stabilizers especially useful herein preferably physically adhere on, or associate with, the surface of the nanoparticulate vitamin K2 particles, but do not chemically react with the vitamin K2 particles or itself. Individually adsorbed molecules of the surface stabilizer are essentially free of intermolecular cross-linkages.

The present invention also includes vitamin K2 compositions together with one or more non-toxic physiologically acceptable carriers, adjuvants, or vehicles, collectively referred to as carriers. The compositions can be formulated for parenteral injection (e.g., intravenous, intramuscular, or subcutaneous), oral administration in solid, liquid, or aerosol form, vaginal, nasal, rectal, ocular, local (powders, ointments or drops), buccal, intracisternal, intraperitoneal, or topical administration, and the like.

1. Vitamin K2

The compositions of the invention comprise particles of vitamin K2, or a polymorph or variant thereof. The vitamin K2 particles can be crystalline, semi-crystalline, amorphous, semi-amorphous, or a combination thereof.

2. Surface Stabilizers

The choice of a surface stabilizer for a vitamin K2 is non-trivial and required extensive experimentation to realize a desirable formulation. Accordingly, the present invention is directed to the surprising discovery that nanoparticulate vitamin K2 compositions can be made.

Combinations of more than one surface stabilizers can be used in the invention. Useful surface stabilizers which can be employed in the invention include, but are not limited to, known organic and inorganic pharmaceutical excipients. Such excipients include various polymers, low molecular weight oligomers, natural products, and surfactants. Exemplary surface stabilizers include nonionic, anionic, cationic, ionic, and zwitterionic surfactants.

Representative examples of surface stabilizers include hydroxypropyl methylcellulose (now known as hypromellose), hydroxypropylcellulose, polyvinylpyrrolidone, sodium lauryl sulfate, dioctylsulfosuccinate, gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens® such as e.g., Tween 20® and Tween 80® (ICI Speciality Chemicals)); polyethylene glycols (e.g., Carbowaxs 3550® and 934® (Union Carbide)), polyoxyethylene stearates, colloidal silicon dioxide, phosphates, carboxymethylcellulose calcium, carboxymethylcellulose sodium, methylcellulose, hydroxyethylcellulose, hypromellose phthalate, noncrystalline cellulose, magnesium aluminium silicate, triethanolamine, polyvinyl alcohol (PVA), 4-(1,1,3,3-tetramethylbutyl)-phenol polymer with ethylene oxide and formaldehyde (also known as tyloxapol, superione, and triton), poloxamers (e.g., Pluronics F68® and F108®, which are block copolymers of ethylene oxide and propylene oxide); poloxamines (e.g., Tetronic 908®, also known as Poloxamine 908®, which is a tetrafunctional block copolymer derived from sequential addition of propylene oxide and ethylene oxide to ethylenediamine (BASF Wyandotte Corporation, Parsippany, N.J.)); Tetronic 1508® (T-1508) (BASF Wyandotte Corporation), Tritons X-200®, which is an alkyl aryl polyether sulfonate (Rohm and Haas); Crodestas F-110®, which is a mixture of sucrose stearate and sucrose distearate (Croda Inc.); p-isononylphenoxypoly-(glycidol), also known as Olin-10G® or Surfactant 10-G® (Olin Chemicals, Stamford, Conn.); Crodestas SL-40® (Croda, Inc.); and SA9OHCO, which is C₁₈H₃₇CH₂(CON(CH₃)—CH₂(CHOH)₄(CH₂0H)₂ (Eastman Kodak Co.); decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; PEG-phospholipid, PEG-cholesterol, PEG-cholesterol derivative, PEG-vitamin A, PEG-vitamin E, lysozyme, random copolymers of vinyl pyrrolidone and vinyl acetate, and the like.

Examples of useful cationic surface stabilizers include, but are not limited to, polymers, biopolymers, polysaccharides, cellulosics, alginates, phospholipids, and nonpolymeric compounds, such as zwitterionic stabilizers, poly-n-methylpyridinium, anthryul pyridinium chloride, cationic phospholipids, chitosan, polylysine, polyvinylimidazole, polybrene, polymethylmethacrylate trimethylammoniumbromide bromide (PMMTMABr), hexyldesyltrimethylammonium bromide (HDMAB), and polyvinylpyrrolidone-2-dimethylaminoethyl methacrylate dimethyl sulfate.

Other useful cationic stabilizers include, but are not limited to, cationic lipids, sulfonium, phosphonium, and quarternary ammonium compounds, such as stearyltrimethylammonium chloride, benzyl-di(2-chloroethyl)ethylammonium bromide, coconut trimethyl ammonium chloride or bromide, coconut methyl dihydroxyethyl ammonium chloride or bromide, decyl triethyl ammonium chloride, decyl dimethyl hydroxyethyl ammonium chloride or bromide, C₁₂₋₁₅dimethyl hydroxyethyl ammonium chloride or bromide, coconut dimethyl hydroxyethyl ammonium chloride or bromide, myristyl trimethyl ammonium methyl sulphate, lauryl dimethyl benzyl ammonium chloride or bromide, lauryl dimethyl (ethenoxy)₄ ammonium chloride or bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts and dialkyl-dimethylammonium salts, lauryl trimethyl ammonium chloride, ethoxylated alkyamidoalkyldialkylammonium salt and/or an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride and dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂, C₁₅, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride (ALIQUAT 336™), POLYQUAT 10™, tetrabutylammonium bromide, benzyl trimethylammonium bromide, choline esters (such as choline esters of fatty acids), benzalkonium chloride, stearalkonium chloride compounds (such as stearyltrimonium chloride and Distearyldimonium chloride), cetyl pyridinium bromide or chloride, halide salts of quaternized polyoxyethylalkylamines, MIRAPOL™ and ALKAQUATT™ (Alkaril Chemical Company), alkyl pyridinium salts; amines, such as alkylamines, dialkylamines, alkanolamines, polyethylenepolyamines, N,N-dialkylaminoalkyl acrylates, and vinyl pyridine, amine salts, such as lauryl amine acetate, stearyl amine acetate, alkylpyridinium salt, and alkylimidazolium salt, and amine oxides; imide azolinium salts; protonated quaternary acrylamides; methylated quaternary polymers, such as poly[diallyl dimethylammonium chloride] and poly-[N-methyl vinyl pyridinium chloride]; and cationic guar.

Such exemplary cationic surface stabilizers and other useful cationic surface stabilizers are described in J. Cross and E. Singer, Cationic Surfactants: Analytical and Biological Evaluation (Marcel Dekker, 1994); P. and D. Rubingh (Editor), Cationic Surfactants: Physical Chemistry (Marcel Dekker, 1991); and J. Richmond, Cationic Surfactants: Organic Chemistry, (Marcel Dekker, 1990).

Nonpolymeric surface stabilizers are any nonpolymeric compound, such benzalkonium chloride, a carbonium compound, a phosphonium compound, an oxonium compound, a halonium compound, a cationic organometallic compound, a quarternary phosphorous compound, a pyridinium compound, an anilinium compound, an ammonium compound, a hydroxylammonium compound, a primary ammonium compound, a secondary ammonium compound, a tertiary ammonium compound, and quarternary ammonium compounds of the formula NR₁R₂R₃R₄⁽⁺⁾. For compounds of the formula NR₁R₂R₃R₄⁽⁺⁾:
(i) none of R₁-R₄ are CH₃;
(ii) one of R₁-R₄ is CH₃;
(iii) three of R₁-R₄ are CH₃;
(iv) all of R₁-R₄ are CH₃;
(v) two of R₁-R₄ are CH₃, one of R₁-R₄ is C₆H₅CH₂, and one of R₁-R₄ is an alkyl chain of seven carbon atoms or less;
(vi) two of R₁-R₄ are CH₃, one of R₁-R₄ is C₆H₅CH₂, and one of R₁-R₄ is an alkyl chain of nineteen carbon atoms or more;
(vii) two of R₁-R₄ are CH₃ and one of R₁-R₄ is the group C₆H₅(CH₂)ₙ, where n>1;
(viii) two of R₁-R₄ are CH₃, one of R₁-R₄ is C₆H₅CH₂, and one of R₁-R₄ comprises at least one heteroatom;
(ix) two of R₁-R₄ are CH₃, one of R₁-R₄ is C₆H₅CH₂, and one of R₁-R₄ comprises at least one halogen;
(x) two of R₁-R₄ are CH₃, one of R₁-R₄ is C₆H₅CH₂, and one of R₁-R₄ comprises at least one cyclic fragment;
(xi) two of R₁-R₄ are CH₃ and one of R₁-R₄ is a phenyl ring; or
(xii) two of R₁-R₄ are CH₃ and two of R₁-R₄ are purely aliphatic fragments.

Such compounds include, but are not limited to, behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride (Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3) oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide.

The surface stabilizers are commercially available and/or can be prepared by techniques known in the art. Most of these surface stabilizers are known pharmaceutical excipients and are described in detail in the Handbook of Pharmaceutical Excipients, published jointly by the American Pharmaceutical Association and The Pharmaceutical Society of Great Britain (The Pharmaceutical Press, 2000), specifically incorporated by reference.

3. Other Pharmaceutical Excipients

Pharmaceutical compositions according to the invention may also comprise one or more binding agents, filling agents, lubricating agents, suspending agents, sweeteners, flavoring agents, preservatives, buffers, wetting agents, disintegrants, effervescent agents, and other excipients. Such excipients are known in the art.

Examples of filling agents are lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCCT™).

Suitable lubricants, including agents that act on the flowability of the powder to be compressed, are colloidal silicon dioxide, such as Aerosil® 200, talc, stearic acid, magnesium stearate, calcium stearate, and silica gel.

Examples of sweeteners are any natural or artificial sweetener, such as sucrose, xylitol, sodium saccharin, cyclamate, aspartame, and acsulfame. Examples of flavoring agents are Magnasweet® (trademark of MAFCO), bubble gum flavor, and fruit flavors, and the like.

Examples of preservatives are potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quarternary compounds such as benzalkonium chloride.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel® PH101 and Avicel PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

4. Nanoparticulate Vitamin K2 Particle Size

The compositions of the invention comprise nanoparticulate vitamin K2 particles which have an effective average particle size of less than about 2000 nm (i.e., 2 microns), less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods. Preferably, at least about 70%, about 90%, or about 95% of the vitamin K2 particles have a particle size of less than the effective average, i.e., less than about 2000 nm, 1900 nm, 1800 nm, 1700 nm, etc.

In the present invention, the value for D50 of a nanoparticulate vitamin K2 composition is the particle size below which 50% of the vitamin K2 particles fall, by weight. Similarly, D90 is the particle size below which 90% of the vitamin K2 particles fall, by weight.

5. Concentration of Vitamin K2 and Surface Stabilizers

The relative amounts of vitamin K2 and one or more surface stabilizers can vary widely. The optimal amount of the individual components can depend, for example, upon the particular vitamin K2 selected, the hydrophilic lipophilic balance (HLB), melting point, and the surface tension of water solutions of the stabilizer, etc.

The concentration of the vitamin K2 can vary from about 99.5% to about 0.001%, from about 95% to about 0.1%, or from about 90% to about 0.5%, by weight, based on the total combined weight of the vitamin K2 and at least one surface stabilizer, not including other excipients.

The concentration of the at least one surface stabilizer can vary from about 0.5% to about 99.999%, from about 5.0% to about 99.9%, or from about 10% to about 99.5%, by weight, based on the total combined dry weight of the vitamin K2 and at least one surface stabilizer, not including other excipients.

6. Exemplary Nanoparticulate Vitamin K2 Tablet Formulations

Several exemplary vitamin K2 tablet formulations are given below. These examples are not intended to limit the claims in any respect, but rather to provide exemplary tablet formulations of vitamin K2 which can be utilized in the methods of the invention. Such exemplary tablets can also comprise a coating agent.

TABLE 1
Exemplary Nanoparticulate
Vitamin K2 Tablet Formulation #1

| Component | g/Kg |
|---|---|
| Vitamin K2 | about 50 to about 500 |
| Hypromellose, USP | about 10 to about 70 |
| Docusate Sodium, USP | about 1 to about 10 |
| Sucrose, NF | about 100 to about 500 |
| Sodium Lauryl Sulfate, NF | about 1 to about 40 |
| Lactose Monohydrate, NF | about 50 to about 400 |
| Silicified Microcrystalline Cellulose | about 50 to about 300 |
| Crospovidone, NF | about 20 to about 300 |
| Magnesium Stearate, NF | about 0.5 to about 5 |

TABLE 2
Exemplary Nanoparticulate
Vitamin K2 Tablet Formulation #2

| Component | g/Kg |
|---|---|
| Vitamin K2 | about 100 to about 300 |
| Hypromellose, USP | about 30 to about 50 |
| Docusate Sodium, USP | about 0.5 to about 10 |
| Sucrose, NF | about 100 to about 300 |
| Sodium Lauryl Sulfate, NF | about 1 to about 30 |
| Lactose Monohydrate, NF | about 100 to about 300 |
| Silicified Microcrystalline Cellulose | about 50 to about 200 |
| Crospovidone, NF | about 50 to about 200 |
| Magnesium Stearate, NF | about 0.5 to about 5 |

TABLE 3
Exemplary Nanoparticulate
Vitamin K2 Tablet Formulation #3

| Component | g/Kg |
|---|---|
| Vitamin K2 | about 200 to about 225 |
| Hypromellose, USP | about 42 to about 46 |
| Docusate Sodium, USP | about 2 to about 6 |
| Sucrose, NF | about 200 to about 225 |
| Sodium Lauryl Sulfate, NF | about 12 to about 18 |
| Lactose Monohydrate, NF | about 200 to about 205 |
| Silicified Microcrystalline Cellulose | about 130 to about 135 |
| Crospovidone, NF | about 112 to about 118 |
| Magnesium Stearate, NF | about 0.5 to about 3 |

TABLE 4
Exemplary Nanoparticulate
Vitamin K2 Tablet Formulation #4

| Component | g/Kg |
|---|---|
| Vitamin K2 | about 119 to about 224 |
| Hypromellose, USP | about 42 to about 46 |
| Docusate Sodium, USP | about 2 to about 6 |
| Sucrose, NF | about 119 to about 224 |
| Sodium Lauryl Sulfate, NF | about 12 to about 18 |
| Lactose Monohydrate, NF | about 119 to about 224 |
| Silicified Microcrystalline Cellulose | about 129 to about 134 |
| Crospovidone, NF | about 112 to about 118 |
| Magnesium Stearate, NF | about 0.5 to about 3 |

### D. Methods of Making Nanoparticulate Vitamin K2 Compositions

The nanoparticulate vitamin K2 compositions can be made using, for example, milling, homogenization, precipitation, freezing or supercritical fluids, or template emulsion techniques. Exemplary methods of making nanoparticulate compositions are described in the '684 patent. Methods of making Methods of making nanoparticulate compositions are also described in U.S. Pat. No. 5,518,187 for “Method of Grinding Pharmaceutical Substances;” U.S. Pat. No. 5,718,388 for “Continuous Method of Grinding Pharmaceutical Substances;” U.S. Pat. No. 5,862,999 for “Method of Grinding Pharmaceutical Substances;” U.S. Pat. No. 5,665,331 for “Co-Microprecipitation of Nanoparticulate Pharmaceutical Agents with Crystal Growth Modifiers;” U.S. Pat. No. 5,662,883 for “Co-Microprecipitation of Nanoparticulate Pharmaceutical Agents with Crystal Growth Modifiers;” U.S. Pat. No. 5,560,932 for “Microprecipitation of Nanoparticulate Pharmaceutical Agents;” U.S. Pat. No. 5,543,133 for “Process of Preparing X-Ray Contrast Compositions Containing Nanoparticles;” U.S. Pat. No. 5,534,270 for “Method of Preparing Stable Drug Nanoparticles;” U.S. Pat. No. 5,510,118 for “Process of Preparing Therapeutic Compositions Containing Nanoparticles;” and U.S. Pat. No. 5,470,583 for “Method of Preparing Nanoparticle Compositions Containing Charged Phospholipids to Reduce Aggregation,” all of which are specifically incorporated by reference.

The resultant nanoparticulate vitamin K2 compositions or dispersions can be utilized in solid or liquid dosage formulations, such as liquid dispersions, gels, aerosols, ointments, creams, controlled release formulations, fast melt formulations, lyophilized formulations, tablets, capsules, delayed release formulations, extended release formulations, pulsatile release formulations, mixed immediate release and controlled release formulations, etc.

1. Milling to Obtain Nanoparticulate Vitamin K2 Dispersions

Milling a vitamin K2 to obtain a nanoparticulate dispersion comprises dispersing the vitamin K2 particles in a liquid dispersion medium in which the vitamin K2 is poorly soluble, followed by applying mechanical means in the presence of grinding media to reduce the particle size of the vitamin K2 to the desired effective average particle size. The dispersion medium can be, for example, water, safflower oil, ethanol, t-butanol, glycerin, polyethylene glycol (PEG), hexane, or glycol. A preferred dispersion medium is water.

The vitamin K2 particles can be reduced in size in the presence of at least one surface stabilizer. Alternatively, vitamin K2 particles can be contacted with one or more surface stabilizers after attrition. Other compounds, such as a diluent, can be added to the vitamin K2/surface stabilizer composition during the size reduction process. Dispersions can be manufactured continuously or in a batch mode.

One of skill in the art would understand that it may be the case that, following milling, not all particles may be reduced to the desired size. In such an event, the particles of the desired size may be separated and used in the practice of the present invention.

2. Precipitation to Obtain Nanoparticulate Vitamin K2 Compositions

Another method of forming the desired nanoparticulate vitamin K2 composition is by microprecipitation. This is a method of preparing stable dispersions of poorly soluble active agents in the presence of one or more surface stabilizers and one or more colloid stability enhancing surface active agents free of any trace toxic solvents or solubilized heavy metal impurities. Such a method comprises, for example: (1) dissolving the vitamin K2 in a suitable solvent; (2) adding the formulation from step (1) to a solution comprising at least one surface stabilizer; and (3) precipitating the formulation from step (2) using an appropriate non-solvent. The method can be followed by removal of any formed salt, if present, by dialysis or diafiltration and concentration of the dispersion by conventional means.

3. Homogenization to Obtain Nanoparticulate Vitamin K2 Compositions

Exemplary homogenization methods of preparing active agent nanoparticulate compositions are described in U.S. Pat. No. 5,510,118, for “Process of Preparing Therapeutic Compositions Containing Nanoparticles.” Such a method comprises dispersing particles of a vitamin K2 in a liquid dispersion medium, followed by subjecting the dispersion to homogenization to reduce the particle size of a vitamin K2 to the desired effective average particle size. The vitamin K2 particles can be reduced in size in the presence of at least one surface stabilizer. Alternatively, the vitamin K2 particles can be contacted with one or more surface stabilizers either before or after attrition. Other compounds, such as a diluent, can be added to the vitamin K2/surface stabilizer composition either before, during, or after the size reduction process. Dispersions can be manufactured continuously or in a batch mode.

4. Cryogenic Methodologies to Obtain Nanoparticulate Vitamin K2 Compositions

Another method of forming the desired nanoparticulate vitamin K2 composition is by spray freezing into liquid (SFL). This technology comprises an organic or organoaqueous solution of vitamin K2 with stabilizers, which is injected into a cryogenic liquid, such as liquid nitrogen. The droplets of the vitamin K2 solution freeze at a rate sufficient to minimize crystallization and particle growth, thus formulating nanostructured vitamin K2 particles. Depending on the choice of solvent system and processing conditions, the nanoparticulate vitamin K2 particles can have varying particle morphology. In the isolation step, the nitrogen and solvent are removed under conditions that avoid agglomeration or ripening of the vitamin K2 particles.

As a complementary technology to SFL, ultra rapid freezing (URF) may also be used to create equivalent nanostructured vitamin K2 particles with greatly enhanced surface area. URF comprises taking a water-miscible, anhydrous, organic, or organoaqueous solution of vitamin K2 with stabilizers and applying it onto a cryogenic substrate. The solvent is then removed by means such as lyophilization or atmospheric freeze-drying with the resulting nanostructured vitamin K2 remaining.

5. Emulsion Methodologies to Obtain Nanoparticulate Vitamin K2 Compositions

Another method of forming the desired nanoparticulate vitamin K2 composition is by template emulsion. Template emulsion creates nanostructured vitamin K2 particles with controlled particle size distribution and rapid dissolution performance. The method comprises an oil-in-water emulsion that is prepared, then swelled with a non-aqueous solution comprising the vitamin K2 and stabilizers. The particle size distribution of the vitamin K2 particles is a direct result of the size of the emulsion droplets prior to loading with the vitamin K2 a property which can be controlled and optimized in this process. Furthermore, through selected use of solvents and stabilizers, emulsion stability is achieved with no or suppressed Ostwald ripening. Subsequently, the solvent and water are removed, and the stabilized nanostructured vitamin K2 particles are recovered. Various vitamin K2 particles morphologies can be achieved by appropriate control of processing conditions.

### E. Methods of Using the Nanoparticulate Vitamin K2 Compositions of the Invention

The invention provides a method of increasing bioavailability of a vitamin K2 in a subject. Such a method comprises orally administering to a subject an effective amount of a composition comprising a vitamin K2. The vitamin K2 composition, in accordance with standard pharmacokinetic practice, has a bioavailability that is about 50% greater than a conventional dosage form, about 40% greater, about 30% greater, about 20% or about 10% greater.

The compositions of the invention are useful in the prevention and treatment of osteoporosis.

The vitamin K2 compounds of the invention can be administered to a subject via any conventional means including, but not limited to, orally, rectally, ocularly, parenterally (e.g., intravenous, intramuscular, or subcutaneous), intracistemally, pulmonary, intravaginally, intraperitoneally, locally (e.g., powders, ointments or drops), or as a buccal or nasal spray. As used herein, the term “subject” is used to mean an animal, preferably a mammal, including a human or non-human. The terms patient and subject may be used interchangeably.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents, or vehicles including water, ethanol, polyols (propyleneglycol, polyethylene-glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

The nanoparticulate vitamin K2 compositions may also contain adjuvants such as preserving, wetting, emulsifying, and dispensing agents. Prevention of the growth of microorganisms can be ensured by various antibacterial and antifungal agents, such as parabens, chlorobutanol, phenol, sorbic acid, and the like. It may also be desirable to include isotonic agents, such as sugars, sodium chloride, and the like. Prolonged absorption of the injectable pharmaceutical form can be brought about by the use of agents delaying absorption, such as aluminum monostearate and gelatin.

Solid dosage forms for oral administration include, but are not limited to, capsules, tablets, pills, powders, and granules. In such solid dosage forms, the active agent is admixed with at least one of the following: (a) one or more inert excipients (or carriers), such as sodium citrate or dicalcium phosphate; (b) fillers or extenders, such as starches, lactose, sucrose, glucose, mannitol, and silicic acid; (c) binders, such as carboxymethylcellulose, alignates, gelatin, polyvinylpyrrolidone, sucrose, and acacia; (d) humectants, such as glycerol; (e) disintegrating agents, such as agar-agar, calcium carbonate, potato or tapioca starch, alginic acid, certain complex silicates, and sodium carbonate; (f) solution retarders, such as paraffin; (g) absorption accelerators, such as quaternary ammonium compounds; (h) wetting agents, such as cetyl alcohol and glycerol monostearate; (i) adsorbents, such as kaolin and bentonite; and (j) lubricants, such as talc, calcium stearate, magnesium stearate, solid polyethylene glycols, sodium lauryl sulfate, or mixtures thereof. For capsules, tablets, and pills, the dosage forms may also comprise buffering agents.

Liquid dosage forms for oral administration include pharmaceutically acceptable emulsions, solutions, suspensions, syrups, and elixirs. In addition to a vitamin K2, the liquid dosage forms may comprise inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, oils, such as cottonseed oil, groundnut oil, corn germ oil, olive oil, castor oil, and sesame oil, glycerol, tetrahydrofurfuryl alcohol, polyethyleneglycols, fatty acid esters of sorbitan, or mixtures of these substances, and the like.

Besides such inert diluents, the composition can also include adjuvants, such as wetting agents, emulsifying and suspending agents, sweetening, flavoring, and perfuming agents.

“Therapeutically effective amount” as used herein with respect to a vitamin K2, dosage shall mean that dosage that provides the specific pharmacological response for which a vitamin K2 is administered in a significant number of subjects in need of such treatment. It is emphasized that ‘therapeutically effective amount,’ administered to a particular subject in a particular instance will not always be effective in treating the diseases described herein, even though such dosage is deemed a ‘therapeutically effective amount’ by those skilled in the art. It is to be further understood that vitamin K2 dosages are, in particular instances, measured as oral dosages, or with reference to drug levels as measured in blood.

One of ordinary skill will appreciate that effective amounts of a vitamin K2 can be determined empirically and can be employed in pure form or, where such forms exist, in pharmaceutically acceptable salt, ester, or prodrug form. Actual dosage levels of a vitamin K2 in the nanoparticulate compositions of the invention may be varied to obtain an amount of a vitamin K2 that is effective to obtain a desired therapeutic response for a particular composition and method of administration. The selected dosage level therefore depends upon the desired therapeutic effect, the route of administration, the potency of the administered vitamin K2, the desired duration of treatment, and other factors.

Dosage unit compositions may contain such amounts of such submultiples thereof as may be used to make up the daily dose. It will be understood, however, that the specific dose level for any particular patient will depend upon a variety of factors: the type and degree of the cellular or physiological response to be achieved; activity of the specific agent or composition employed; the specific agents or composition employed; the age, body weight, general health, sex, and diet of the patient; the time of administration, route of administration, and rate of excretion of the agent; the duration of the treatment; drugs used in combination or coincidental with the specific agent; and like factors well known in the medical arts.

### II. Controlled Release Vitamin K2 Compositions

The effectiveness of pharmaceutical compounds in the prevention and treatment of disease states depends on a variety of factors including the rate and duration of delivery of the compound from the dosage form to the patient. The combination of delivery rate and duration exhibited by a given dosage form in a patient can be described as its in vivo release profile and, depending on the pharmaceutical compound administered, will be associated with a concentration and duration of the pharmaceutical compound in the blood plasma, referred to as a plasma profile. As pharmaceutical compounds vary in their pharmacokinetic properties such as bioavailability, and rates of absorption and elimination, the release profile and the resultant plasma profile become important elements to consider in designing effective drug therapies.

The release profiles of dosage forms may exhibit different rates and durations of release and may be continuous or pulsatile. Continuous release profiles include release profiles in which a quantity of one or more pharmaceutical compounds is released continuously throughout the dosing interval at either a constant or variable rate. Pulsatile release profiles include release profiles in which at least two discrete quantities of one or more pharmaceutical compounds are released at different rates and/or over different time frames. For any given pharmaceutical compound or combination of such compounds, the release profile for a given dosage form gives rise to an associated plasma profile in a patient. When two or more components of a dosage form have different release profiles, the release profile of the dosage form as a whole is a combination of the individual release profiles and may be described generally as “multimodal.” The release profile of a two-component dosage form in which each component has a different release profile may described as “bimodal,” and the release profile of a three-component dosage form in which each component has a different release profile may described as “trimodal.”

Similar to the variables applicable to the release profile, the associated plasma profile in a patient may exhibit constant or variable blood plasma concentration levels of the pharmaceutical compounds over the duration of action and may be continuous or pulsatile. Continuous plasma profiles include plasma profiles of all rates and duration which exhibit a single plasma concentration maximum. Pulsatile plasma profiles include plasma profiles in which at least two higher blood plasma concentration levels of pharmaceutical compound are separated by a lower blood plasma concentration level and may be described generally as “multimodal.” Pulsatile plasma profiles exhibiting two peaks may be described as “bimodal” and plasma profiles exhibiting three peaks may be described as “trimodal.” Depending on, at least in part, the pharmacokinetics of the pharmaceutical compounds included in the dosage form as well as the release profiles of the individual components of the dosage form, a multimodal release profile may result in either a continuous or a pulsatile plasma profile upon administration to a patient.

In one embodiment the present invention provides a multiparticulate modified release composition which delivers vitamin K2 in a pulsatile manner.

In still another embodiment the present invention provides a multiparticulate modified release composition which delivers vitamin K2 in a continuous manner.

In yet another embodiment the present invention provides a multiparticulate modified release composition in which a first portion of vitamin K2 is released immediately upon administration and one or more subsequent portions of vitamin K2 are released after an initial time delay.

In yet another embodiment the present invention provides solid oral dosage forms for once-daily or twice-daily administration comprising the multiparticulate modified release composition of the present invention.

In yet another embodiment the present invention provides a multiparticulate modified release composition in which the particles comprise vitamin K2-containing nanoparticles of the type described above.

In still another embodiment the present invention provides a method for the prevention and/or treatment of osteoporosis comprising the administration of a composition of the present invention.

According to one aspect of the present invention, there is provided a pharmaceutical composition having a first component comprising active ingredient-containing particles, and at least one subsequent component comprising active ingredient-containing particles, each subsequent component having a rate and/or duration of release different from the first component wherein at least one of said components comprises vitamin K2-containing particles. The vitamin K2-containing particles may be coated with a modified release coating. Alternatively or additionally, the vitamin K2-containing particles may comprise a modified release matrix material. Following oral delivery, the composition delivers vitamin K2 in a pulsatile manner. In one embodiment, the first component provides an immediate release of vitamin K2 and the one or more subsequent components provide a modified release of vitamin K2. In such embodiments, the immediate release component serves to hasten the onset of action by minimizing the time from administration to a therapeutically effective plasma concentration level, and the one or more subsequent components serve to minimize the variation in plasma concentration levels and/or maintain a therapeutically effective plasma concentration throughout the dosing interval.

The modified release coating and/or the modified release matrix material cause a lag time between the release of the active ingredient from the first population of active ingredient-containing particles and the release of the active ingredient from subsequent populations of active ingredient-containing particles. Where more than one population of active ingredient-containing particles provide a modified release, the modified release coating and/or the modified release matrix material causes a lag time between the release of the active ingredient from the different populations of active ingredient-containing particles. The duration of these lag times may be varied by altering the composition and/or the amount of the modified release coating and/or altering the composition and/or amount of modified release matrix material utilized. Thus, the duration of the lag time can be designed to mimic a desired plasma profile.

Because the plasma profile produced by the modified release composition upon administration is substantially similar to the plasma profile produced by the administration of two or more IR dosage forms given sequentially, the modified release composition of the present invention is particularly useful for administering a vitamin K2 which is normally administered three times daily. In one embodiment of the present invention, the composition delivers the vitamin K2 in a trimodal manner. Upon administration, such a composition produces a plasma profile which substantially mimics that obtained by the sequential administration of three IR doses of vitamin K2 in accordance with a typical treatment regimen.

According to another aspect of the present invention, the composition can be designed to produce a plasma profile that minimizes or eliminates the variations in plasma concentration levels associated with the administration of two or more IR dosage forms given sequentially. In such embodiments, the composition may be provided with an immediate release component to hasten the onset of action by minimizing the time from administration to a therapeutically effective plasma concentration level, and at least one modified release component to maintain a therapeutically effective plasma concentration level throughout the dosing interval.

The term “particulate” as used herein refers to a state of matter which is characterized by the presence of discrete particles, pellets, beads or granules irrespective of their size, shape or morphology. The term “multiparticulate” as used herein means a plurality of discrete or aggregated particles, pellets, beads, granules, or mixtures thereof, irrespective of their size, shape or morphology.

The term “modified release” as used herein includes a release which is not immediate and includes controlled release, extended release, sustained release and delayed release.

The term “time delay” as used herein refers to the period of time between the administration of a dosage form comprising the composition of the invention and the release of the active ingredient from a particular component thereof.

The term “lag time” as used herein refers to the time between the release of the active ingredient from one component of the composition and the release of the active ingredient from another component of the composition.

The term “erodable” as used herein refers to formulations which may be worn away, diminished, or deteriorated by the action of substances within the body.

The term “diffusion controlled” as used herein refers to formulations which may spread as the result of their spontaneous movement, for example, from a region of higher to one of lower concentration.

The term “osmotic controlled” as used herein refers to formulations which may spread as the result of their movement through a semi-permeable membrane into a solution of higher concentration that tends to equalize the concentrations of the formulation on the two sides of the membrane.

The active ingredients in each component may be the same or different. For example, the composition may comprise components comprising only vitamin K2 as the active ingredient. Alternatively, the composition may comprise a first component comprising vitamin K2 and at least one subsequent component comprising an active ingredient other than vitamin K2 suitable for coadministration with vitamin K2, or a first component containing an active ingredient other than vitamin K2 and at least one subsequent component comprising vitamin K2. Indeed, two or more active ingredients may be incorporated into the same component when the active ingredients are compatible with each other. An active ingredient present in one component of the composition may be accompanied by, for example, an enhancer compound or a sensitizer compound in another component of the composition, in order to modify the bioavailability or therapeutic effect thereof.

As used herein, the term “enhancer” refers to a compound which is capable of enhancing the absorption and/or bioavailability of an active ingredient by promoting net transport across the GIT in an animal, such as a human. Enhancers include but are not limited to medium chain fatty acids; salts, esters, ethers and derivatives thereof, including glycerides and triglycerides; non-ionic surfactants such as those that can be prepared by reacting ethylene oxide with a fatty acid, a fatty alcohol, an alkylphenol or a sorbitan or glycerol fatty acid ester; cytochrome P450 inhibitors, P-glycoprotein inhibitors and the like; and mixtures of two or more of these agents.

In those embodiments in which more than one vitamin K2-containing component is present, the proportion of vitamin K2 contained in each component may be the same or different depending on the desired dosing regime. The vitamin K2 present in the first component and in subsequent components may be any amount sufficient to produce a therapeutically effective plasma concentration level. The vitamin K2, when applicable, may be present either in the form of one substantially optically pure stereoisomer or as a mixture, racemic or otherwise, of two or more stereoisomers. The vitamin K2 is preferably present in the composition in an amount of from about 0.1 to about 500 mg, preferably in the amount of from about 1 to about 100 mg. The vitamin K2 is preferably present in the first component in an amount of from about 0.5 to about 60 mg; more preferably the vitamin K2, is present in the first component in an amount of from about 2.5 to about 30 mg. The vitamin K2 is present in subsequent components in an amount within similar ranges to those described for the first component.

The time release characteristics for the delivery of the vitamin K2 from each of the components may be varied by modifying the composition of each component, including modifying any of the excipients and/or coatings which may be present. In particular, the release of the vitamin K2 may be controlled by changing the composition and/or the amount of the modified release coating on the particles, if such a coating is present. If more than one modified release component is present, the modified release coating for each of these components may be the same or different. Similarly, when modified release is facilitated by the inclusion of a modified release matrix material, release of the active ingredient may be controlled by the choice and amount of modified release matrix material utilized. The modified release coating may be present, in each component, in any amount that is sufficient to yield the desired delay time for each particular component. The modified release coating may be preset, in each component, in any amount that is sufficient to yield the desired time lag between components.

The lag time and/or time delay for the release of the vitamin K2 from each component may also be varied by modifying the composition of each of the components, including modifying any excipients and coatings which may be present. For example, the first component may be an immediate release component wherein the vitamin K2 is released immediately upon administration. Alternatively, the first component may be, for example, a time-delayed immediate release component in which the vitamin K2 is released substantially in its entirety immediately after a time delay. The second and subsequent component may be, for example, a time-delayed immediate release component as just described or, alternatively, a time-delayed sustained release or extended release component in which the vitamin K2 is released in a controlled fashion over an extended period of time.

As will be appreciated by those skilled in the art, the exact nature of the plasma concentration curve will be influenced by the combination of all of these factors just described. In particular, the lag time between the delivery (and thus also the onset of action) of the vitamin K2 in each component may be controlled by varying the composition and coating (if present) of each of the components. Thus by variation of the composition of each component (including the amount and nature of the active ingredient(s)) and by variation of the lag time, numerous release and plasma profiles may be obtained. Depending on the duration of the lag time between the release of the vitamin K2 from each component and the nature of the release of the vitamin K2 from each component (i.e. immediate release, sustained release etc.), the plasma profile may be continuous (i.e., having a single maximum) or pulsatile in which the peaks in the plasma profile may be well separated and clearly defined (e.g. when the lag time is long) or superimposed to a degree (e.g. when the lag time is short).

The plasma profile produced from the administration of a single dosage unit comprising the composition of the present invention is advantageous when it is desirable to deliver two or more pulses of active ingredient without the need for administration of two or more dosage units. Additionally, in the case of treating pain and/or inflammation, it is particularly useful to have such a multimodal plasma profile. For example, a typical vitamin K2 treatment regime consists of the administration of two or three doses of an immediate release dosage formulation given four hours apart. This type of regime has been found to be therapeutically effective and is widely used.

Any coating material which modifies the release of the vitamin K2 in the desired manner may be used. In particular, coating materials suitable for use in the practice of the present invention include but are not limited to polymer coating materials, such as cellulose acetate phthalate, cellulose acetate trimaletate, hydroxy propyl methylcellulose phthalate, polyvinyl acetate phthalate, ammonio methacrylate copolymers such as those sold under the trademark Eudragit® RS and RL, poly acrylic acid and poly acrylate and methacrylate copolymers such as those sold under the trademark Eudragit® S and L, polyvinyl acetaldiethylamino acetate, hydroxypropyl methylcellulose acetate succinate, shellac; hydrogels and gel-forming materials, such as carboxyvinyl polymers, sodium alginate, sodium carmellose, calcium carmellose, sodium carboxymethyl starch, polyvinyl alcohol, hydroxyethyl cellulose, methyl cellulose, gelatin, starch, and cellulose based cross-linked polymers—in which the degree of crosslinking is low so as to facilitate adsorption of water and expansion of the polymer matrix, hydroxypropyl cellulose, hydroxypropyl methylcellulose, polyvinylpyrrolidone, crosslinked starch, microcrystalline cellulose, chitin, aminoacryl-methacrylate copolymer (Eudragit® RS-PM, Rohm & Haas), pullulan, collagen, casein, agar, gum arabic, sodium carboxymethyl cellulose, (swellable hydrophilic polymers) poly(hydroxyalkyl methacrylate) (mol. wt. ˜5 k-5,000 k), polyvinylpyrrolidone (mol. wt. ˜10 k-360 k), anionic and cationic hydrogels, polyvinyl alcohol having a low acetate residual, a swellable mixture of agar and carboxymethyl cellulose, copolymers of maleic anhydride and styrene, ethylene, propylene or isobutylene, pectin (mol. wt. ˜0.30 k-300 k), polysaccharides such as agar, acacia, karaya, tragacanth, algins and guar, polyacrylamides, Polyox® polyethylene oxides (mol. wt. ˜100 k-5,000 k), AquaKeep® acrylate polymers, diesters of polyglucan, crosslinked polyvinyl alcohol and poly N-vinyl-2-pyrrolidone, sodium starch glucolate (e.g. Explotab®; Edward Mandell C. Ltd.); hydrophilic polymers such as polysaccharides, methyl cellulose, sodium or calcium carboxymethyl cellulose, hydroxypropyl methyl cellulose, hydroxypropyl cellulose, hydroxyethyl cellulose, nitro cellulose, carboxymethyl cellulose, cellulose ethers, polyethylene oxides (e.g. Polyox®, Union Carbide), methyl ethyl cellulose, ethylhydroxy ethylcellulose, cellulose acetate, cellulose butyrate, cellulose propionate, gelatin, collagen, starch, maltodextrin, pullulan, polyvinyl pyrrolidone, polyvinyl alcohol, polyvinyl acetate, glycerol fatty acid esters, polyacrylamide, polyacrylic acid, copolymers of methacrylic acid or methacrylic acid (e.g. Eudragit®, Rohm and Haas), other acrylic acid derivatives, sorbitan esters, natural gums, lecithins, pectin, alginates, ammonia alginate, sodium, calcium, potassium alginates, propylene glycol alginate, agar, and gums such as arabic, karaya, locust bean, tragacanth, carrageens, guar, xanthan, scleroglucan and mixtures and blends thereof. As will be appreciated by the person skilled in the art, excipients such as plasticisers, lubricants, solvents and the like may be added to the coating. Suitable plasticisers include for example acetylated monoglycerides; butyl phthalyl butyl glycolate; dibutyl tartrate; diethyl phthalate; dimethyl phthalate; ethyl phthalyl ethyl glycolate; glycerin; propylene glycol; triacetin; citrate; tripropioin; diacetin; dibutyl phthalate; acetyl monoglyceride; polyethylene glycols; castor oil; triethyl citrate; polyhydric alcohols, glycerol, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidised tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate.

When the modified release component comprises a modified release matrix material, any suitable modified release matrix material or suitable combination of modified release matrix materials may be used. Such materials are known to those skilled in the art. The term “modified release matrix material” as used herein includes hydrophilic polymers, hydrophobic polymers and mixtures thereof which are capable of modifying the release of a vitamin K2 dispersed therein in vitro or in vivo. Modified release matrix materials suitable for the practice of the present invention include but are not limited to microcrystalline cellulose, sodium carboxymethylcellulose, hydroxyalkylcelluloses such as hydroxypropylmethylcellulose and hydroxypropylcellulose, polyethylene oxide, alkylcelluloses such as methylcellulose and ethylcellulose, polyethylene glycol, polyvinylpyrrolidone, cellulose acetate, cellulose acetate butyrate, cellulose acetate phthalate, cellulose acetate trimellitate, polyvinylacetate phthalate, polyalkylmethacrylates, polyvinyl acetate and mixture thereof.

A modified release composition according to the present invention may be incorporated into any suitable dosage form which facilitates release of the active ingredient in a pulsatile manner. In one embodiment, the dosage form comprises a blend of different populations of active ingredient-containing particles which make up the immediate release and the modified release components, the blend being filled into suitable capsules, such as hard or soft gelatin capsules. Alternatively, the different individual populations of active ingredient-containing particles may be compressed (optionally with additional excipients) into mini-tablets which may be subsequently filled into capsules in the appropriate proportions. Another suitable dosage form is that of a multilayer tablet. In this instance the first component of the modified release composition may be compressed into one layer, with the second component being subsequently added as a second layer of the multilayer tablet. The populations of vitamin K2-containing particles making up the composition of the invention may further be included in rapidly dissolving dosage forms such as an effervescent dosage form or a fast-melt dosage form.

In one embodiment, the composition comprises at least two vitamin K2 components: a first vitamin K2 component and one or more subsequent vitamin K2 components. In such embodiment, the first vitamin K2 component of the composition may exhibit a variety of release profiles including profiles in which substantially all of the vitamin K2 contained in the first component is released rapidly upon administration of the dosage form, released rapidly but after a time delay (delayed release), or released slowly over time. In one such embodiment, the vitamin K2 contained in the first component is released rapidly upon administration to a patient. As used herein, “released rapidly” includes release profiles in which at least about 80% of the active ingredient of a component is released within about an hour after administration, the term “delayed release” includes release profiles in which the active ingredient of a component is released (rapidly or slowly) after a time delay, and the terms “controlled release” and “extended release” include release profiles in which at least about 80% of the active ingredient contained in a component is released slowly.

The second vitamin K2 component of such embodiment may also exhibit a variety of release profiles including an immediate release profile, a delayed release profile or a controlled release profile. In one such embodiment, the second vitamin K2 component exhibits a delayed release profile in which the vitamin K2 of the component is released after a time delay.

The plasma profile produced by the administration of dosage forms of the present invention which comprise an immediate release vitamin K2 component and at least one modified release vitamin K2 component can be substantially similar to the plasma profile produced by the administration of two or more IR dosage forms given sequentially, or to the plasma profile produced by the administration of separate IR and modified release dosage forms. Accordingly, the dosage forms of the present invention can be particularly useful for administering vitamin K2 where the maintenance of pharmacokinetic parameters may be desired but is problematic.

In one embodiment, the composition and the solid oral dosage forms containing the composition release the vitamin K2 such that substantially all of the vitamin K2 contained in the first component is released prior to release of the vitamin K2 from the at least one second component. When the first component comprises an IR component, for example, it is preferable that release of the vitamin K2 from the at least one second component is delayed until substantially all the vitamin K2 in the IR component has been released. Release of the vitamin K2 from the at least one second component may be delayed as detailed above by the use of a modified release coatings and/or a modified release matrix material.

When it is desirable to minimize patient tolerance by providing a dosage regime which facilitates wash-out of a first dose of the vitamin K2 from a patient's system, release of the vitamin K2 from subsequent components may be delayed until substantially all of the vitamin K2 contained in the first component has been released, and further delayed until at least a portion the vitamin K2 released from the first component has been cleared from the patient's system. In one embodiment, release of the vitamin K2 from subsequent components of the composition is substantially, if not completely, delayed for a period of at least about two hours after administration of the composition. In another embodiment, the release of vitamin K2 from subsequent components of the composition is substantially, if not completely, delayed for a period of at least about four hours after administration of the composition.

As described hereinbelow, the present invention also includes various types of modified release systems by which vitamin K2 may be delivered in either a pulsatile or continuous manner. These systems include but are not limited to: films with vitamin K2 in a polymer matrix (monolithic devices); vitamin K2 contained by the polymer (reservoir devices); polymeric colloidal particles or microencapsulates (microparticles, microspheres or nanoparticles) in the form of reservoir and matrix devices; vitamin K2 contained by a polymer containing a hydrophilic and/or leachable additive e.g., a second polymer, surfactant or plasticizer, etc. to give a porous device, or a device in which vitamin K2 release may be osmotically controlled (both reservoir and matrix devices); enteric coatings (ionizable and dissolve at a suitable pH); (soluble) polymers with (covalently) attached pendant drug molecules; and devices where release rate is controlled dynamically: e.g., the osmotic pump.

The delivery mechanism of the present invention can control the rate of release of vitamin K2. While some mechanisms will release vitamin K2 at a constant rate, others will vary as a function of time depending on factors such as changing concentration gradients or additive leaching leading to porosity, etc.

Polymers used in sustained release coatings are necessarily biocompatible, and ideally biodegradable. Examples of both naturally occurring polymers such as Aquacoat® (FMC Corporation, Food & Pharmaceutical Products Division, Philadelphia, USA) (ethylcellulose mechanically spheronised to sub-micron sized, aqueous based, pseudo-latex dispersions), and also synthetic polymers such as the Eudragit® (Rohm Pharma, Weiterstadt.) range of poly(acrylate, methacrylate) copolymers are known in the art.

Reservoir Devices

A typical approach to modified release is to encapsulate or contain the drug entirely (e.g., as a core), within a polymer film or coat (i.e., microcapsules or spray/pan coated cores).

The various factors that can affect the diffusion process may readily be applied to reservoir devices (e.g, the effects of additives, polymer functionality (and, hence, sink-solution pH) porosity, film casting conditions, etc.) and, hence, the choice of polymer must be an important consideration in the development of reservoir devices. Modeling the release characteristics of reservoir devices (and monolithic devices) in which the transport of the drug is by a solution-diffusion mechanism therefore typically involves a solution to Fick's second law (unsteady-state conditions; concentration dependent flux) for the relevant boundary conditions. When the device contains dissolved active agent, the rate of release decreases exponentially with time as the concentration (activity) of the agent (i.e., the driving force for release) within the device decreases (i.e., first order release). If, however, the active agent is in a saturated suspension, then the driving force for release is kept constant until the device is no longer saturated. Alternatively the release-rate kinetics may be desorption controlled, and a function of the square root of time.

Transport properties of coated tablets, may be enhanced compared to free-polymer films, due to the enclosed nature of the tablet core (permeant) which may enable the internal build-up of an osmotic pressure which will then act to force the permeant out of the tablet.

The effect of de-ionized water on salt containing tablets coated in polyethylene glycol) (PEG)-containing silicone elastomer, and also the effects of water on free films has been investigated. The release of salt from the tablets was found to be a mixture of diffusion through water filled pores, formed by hydration of the coating, and osmotic pumping. KCl transport through films containing just 10% PEG was negligible, despite extensive swelling observed in similar free films, indicating that porosity was necessary for the release of the KCl which then occurred by trans-pore diffusion. Coated salt tablets, shaped as disks, were found to swell in de-ionized water and change shape to an oblate spheroid as a result of the build-up of internal hydrostatic pressure: the change in shape providing a means to measure the force generated. As might be expected, the osmotic force decreased with increasing levels of PEG content. The lower PEG levels allowed water to be imbibed through the hydrated polymer, while the porosity resulting from the coating dissolving at higher levels of PEG content (20 to 40%) allow the pressure to be relieved by the flow of KCl.

Methods and equations have been developed, which by monitoring (independently) the release of two different salts (e.g., KCl and NaCl) allowed the calculation of the relative magnitudes that both osmotic pumping and trans-pore diffusion contributed to the release of salt from the tablet. At low PEG levels, osmotic flow was increased to a greater extent than was trans-pore diffusion due to the generation of only a low pore number density: at a loading of 20%, both mechanisms contributed approximately equally to the release. The build-up of hydrostatic pressure, however, decreased the osmotic inflow, and osmotic pumping. At higher loadings of PEG, the hydrated film was more porous and less resistant to outflow of salt. Hence, although the osmotic pumping increased (compared to the lower loading), trans-pore diffusion was the dominant release mechanism. An osmotic release mechanism has also been reported for microcapsules containing a water soluble core.

Monolithic Devices (Matrix Devices)

Monolithic (matrix) devices are commonly used for controlling the release of drugs. This is possibly because they are relatively easy to fabricate compared to reservoir devices, and the danger of an accidental high dosage that could result from the rupture of the membrane of a reservoir device is not present. In such a device, the active agent is present as a dispersion within the polymer matrix, and they are typically formed by the compression of a polymer/drug mixture or by dissolution or melting. The dosage release properties of monolithic devices may be dependent upon the solubility of the drug in the polymer matrix or, in the case of porous matrixes, the solubility in the sink solution within the particle's pore network, and also the tortuosity of the network (to a greater extent than the permeability of the film), dependent on whether the drug is dispersed in the polymer or dissolved in the polymer. For low loadings of drug, (0 to 5% W/V) the drug will be released by a solution-diffusion mechanism (in the absence of pores). At higher loadings (5 to 10% W/V), the release mechanism will be complicated by the presence of cavities formed near the surface of the device as the drug is lost: such cavities fill with fluid from the environment increasing the rate of release of the drug.

It is common to add a plasticizer (e.g., a poly(ethylene glycol)), a surfactant, or adjuvant (i.e., an ingredient which increases effectiveness), to matrix devices (and reservoir devices) as a means to enhance the permeability (although, in contrast, plasticizers may be fugitive, and simply serve to aid film formation and, hence, decrease permeability—a property normally more desirable in polymer paint coatings). It was noted that the leaching of PEG increased the permeability of (ethyl cellulose) films linearly as a function of PEG loading by increasing the porosity, however, the films retained their bather properties, not permitting the transport of electrolyte. It was deduced that the enhancement of their permeability was as a result of the effective decrease in thickness caused by the PEG leaching. This was evidenced from plots of the cumulative permeant flux per unit area as a function of time and film reciprocal thickness at a PEG loading of 50% W/W: plots showing a linear relationship between the rate of permeation and reciprocal film thickness, as expected for a (Fickian) solution-diffusion type transport mechanism in a homogeneous membrane. Extrapolation of the linear regions of the graphs to the time axis gave positive intercepts on the time axis: the magnitude of which decreased towards zero with decreasing film thickness. These changing lag times were attributed to the occurrence of two diffusional flows during the early stages of the experiment (the flow of the drug and also the flow of the PEG), and also to the more usual lag time during which the concentration of permeant in the film is building-up. Caffeine, when used as a permeant, showed negative lag times. No explanation of this was forthcoming, but it was noted that caffeine exhibited a low partition coefficient in the system, and that this was also a feature of aniline permeation through polyethylene films which showed a similar negative time lag.

The effects of added surfactants on (hydrophobic) matrix devices has been investigated. It was thought that surfactant may increase the drug release rate by three possible mechanisms: (i) increased solubilization, (ii) improved ‘wettability’ to the dissolution media, and (iii) pore formation as a result of surfactant leaching. For the system studied (Eudragit® RL 100 and RS 100 plasticised by sorbitol, flurbiprofen as the drug, and a range of surfactants) it was concluded that improved wetting of the tablet led to only a partial improvement in drug release (implying that the release was diffusion, rather than dissolution, controlled), although the effect was greater for Eudragit® RS than Eudragit® RL, while the greatest influence on release was by those surfactants that were more soluble due to the formation of disruptions in the matrix allowing the dissolution medium access to within the matrix. This is of obvious relevance to a study of latex films which might be suitable for pharmaceutical coatings, due to the ease with which a polymer latex may be prepared with surfactant as opposed to surfactant-free. Differences were found between the two polymers with only the Eudragit® RS showing interactions between the anionic/cationic surfactant and drug. This was ascribed to the differing levels of quaternary ammonium ions on the polymer.

Composite devices consisting of a polymer/drug matrix coated in a polymer containing no drug also exist. Such a device was constructed from aqueous Eudragit® lattices, and was found to provide a continuous release by diffusion of the drug from the core through the shell. Similarly, a polymer core containing the drug has been produced and coated with a shell that was eroded by gastric fluid. The rate of release of the drug was found to be relatively linear (a function of the rate limiting diffusion process through the shell) and inversely proportional to the shell thickness, whereas the release from the core alone was found to decrease with time.

Microspheres

Methods for the preparation of hollow microspheres have been described. Hollow microspheres were formed by preparing a solution of ethanol/dichloromethane containing the drug and polymer. On pouring into water, an emulsion is formed containing the dispersed polymer/drug/solvent particles, by a coacervation-type process from which the ethanol rapidly diffused precipitating polymer at the surface of the droplet to give a hard-shelled particle enclosing the drug dissolved in the dichloromethane. A gas phase of dichloromethane was then generated within the particle which, after diffusing through the shell, was observed to bubble to the surface of the aqueous phase. The hollow sphere, at reduced pressure, then filled with water which could be removed by a period of drying. No drug was found in the water. Highly porous matrix-type microspheres have also been described. The matrix-type microspheres were prepared by dissolving the drug and polymer in ethanol. On addition to water, the ethanol diffused from the emulsion droplets to leave a highly porous particle. A suggested use of the microspheres was as floating drug delivery devices for use in the stomach.

Pendent Devices

A means of attaching a range of drugs such as analgesics and antidepressants, etc., by means of an ester linkage to poly(acrylate) ester latex particles prepared by aqueous emulsion polymerization has been developed. These lattices, when passed through an ion exchange resin such that the polymer end groups were converted to their strong acid form, could self-catalyze the release of the drug by hydrolysis of the ester link.

Drugs have been attached to polymers, and also monomers have been synthesized with a pendent drug attached. Dosage forms have been prepared in which the drug is bound to a biocompatible polymer by a labile chemical bond e.g., polyanhydrides prepared from a substituted anhydride (itself prepared by reacting an acid chloride with the drug: methacryloyl chloride and the sodium salt of methoxy benzoic acid) were used to form a matrix with a second polymer (Eudragit® RL) which released the drug on hydrolysis in gastric fluid. The use of polymeric Schiff bases suitable for use as carriers of pharmaceutical amines has also been described.

Enteric Films

Enteric coatings consist of pH sensitive polymers. Typically the polymers are carboxylated and interact very little with water at low pH, while at high pH the polymers ionize causing swelling or dissolving of the polymer. Coatings can therefore be designed to remain intact in the acidic environment of the stomach, protecting either the drug from this environment or the stomach from the drug, but to dissolve in the more alkaline environment of the intestine.

Osmotically Controlled Devices

The osmotic pump is similar to a reservoir device but contains an osmotic agent (e.g., the active agent in salt form) which acts to imbibe water from the surrounding medium via a semi-permeable membrane. Such a device, called an elementary osmotic pump, has been described. Pressure is generated within the device which forces the active agent out of the device via an orifice of a size designed to minimize solute diffusion, while preventing the build-up of a hydrostatic pressure head which can have the effect of decreasing the osmotic pressure and changing the dimensions of the device. While the internal volume of the device remains constant, and there is an excess of solid or saturated solution in the device, then the release rate remains constant delivering a volume equal to the volume of solvent uptake.

Electrically Stimulated Release Devices

Monolithic devices have been prepared using polyelectrolyte gels which swell when, for example, an external electrical stimulus is applied causing a change in pH. The release may be modulated by changes in the applied current to produce a constant or pulsatile release profile.

Hydrogels

In addition to their use in drug matrices, hydrogels find use in a number of biomedical applications such as, for example, soft contact lenses, and various soft implants, and the like.

### Methods of Using Modified Release Vitamin K2 Compositions

According to another aspect of the present invention, there is provided a method for treating a patient suffering from pain and/or inflammation comprising the step of administering a therapeutically effective amount of the vitamin K2 composition of the present invention in solid oral dosage form. Advantages of the method of the present invention include a reduction in the dosing frequency required by conventional multiple IR dosage regimes while still maintaining the benefits derived from a pulsatile plasma profile or eliminating or minimizing the variations in plasma concentration levels. This reduced dosing frequency is advantageous in terms of patient compliance and the reduction in dosage frequency made possible by the method of the present invention would contribute to controlling health care costs by reducing the amount of time spent by health care workers on the administration of drugs.

In the following examples, all percentages are weight by weight unless otherwise stated. The term “purified water” as used throughout the Examples refers to water that has been purified by passing it through a water filtration system. It is to be understood that the examples are for illustrative purposes only, and should not be interpreted as restricting the spirit and breadth of the invention as defined by the scope of the claims that follow.

### Example 1

### Multiparticulate Modified Release Composition Containing Vitamin K2

A multiparticulate modified release composition according to the present invention comprising an immediate release component and a modified release component containing vitamin IC₂ is prepared as follows.

(a) Immediate Release Component.

A solution of vitamin K2 (50:50 racemic mixture) is prepared according to any of the formulations given in Table 1. The methylphenidate solution is then coated onto nonpareil seeds to a level of approximately 16.9% solids weight gain using, for example, a Glatt GPCG3 (Glatt, Protech Ltd., Leicester, UK) fluid bed coating apparatus to form the IR particles of the immediate release component.

TABLE 5
Immediate release component solutions

| | Amount, | % (w/w) |
|---|---|---|
| Ingredient | (i) | (ii) |
| Vitamin K2 | 13.0 | 13.0 |
| Polyethylene Glycol 6000 | 0.5 | 0.5 |
| Polyvinylpyrrolidone | 3.5 | |
| Purified Water | 83.5 | 86.5 |

(b) Modified Release Component

Vitamin K2-containing delayed release particles are prepared by coating immediate release particles prepared according to Example 1(a) above with a modified release coating solution as detailed in Table 2. The immediate release particles are coated to varying levels up to approximately to 30% weight gain using, for example, a fluid bed apparatus.

TABLE 6
Modified release component coating solutions

| | Amount, % (w/w) | | | | | | | |
|---|---|---|---|---|---|---|---|---|
| Ingredient | (i) | (ii) | (iii) | (iv) | (v) | (vi) | (vii) | (viii) |
| Eudragit ® | 49.7 | 42.0 | 47.1 | 53.2 | 40.6 | — | — | 25.0 |
| RS 12.5 | | | | | | | | |
| Eudragit ® | — | — | — | — | — | 54.35 | 46.5 | — |
| S 12.5 | | | | | | | | |
| Eudragit ® | — | | — | — | — | — | — | 25.0 |
| L 12.5 | | | | | | | | |
| Polyvinyl- | — | — | — | 0.35 | 0.3 | — | — | |
| pyrrolidone | | | | | | | | |
| Diethyl- | 0.5 | 0.5 | 0.6 | 1.35 | 0.6 | 1.3 | 1.1 | — |
| phthalate | | | | | | | | |
| Triethyl- | — | — | — | — | — | — | — | 1.25 |
| citrate | | | | | | | | |
| Isopropyl | 39.8 | 33.1 | 37.2 | 45.1 | 33.8 | 44.35 | 49.6 | 46.5 |
| alcohol | | | | | | | | |
| Acetone | 10.0 | 8.3 | 9.3 | — | 8.4 | — | — | — |
| Talc¹ | — | 16.0 | 5.9 | — | 16.3 | — | 2.8 | 2.25 |

¹Talc is simultaneously applied during coating for formulations in column (i), (iv) and (vi).

(c) Encapsulation of Immediate and Delayed Release Particles.

The immediate and delayed release particles prepared according to Example 1(a) and (b) above are encapsulated in size 2 hard gelatin capsules to an overall 20 mg dosage strength using, for example, a Bosch GKF 4000S encapsulation apparatus. The overall dosage strength of 20 mg vitamin K2 was made up of 10 mg from the immediate release component and 10 mg from the modified release component.

### Example 2

### Multiparticulate Modified Release Composition Containing Vitamin K2

Multiparticulate modified release vitamin K2 compositions according to the present invention having an immediate release component and a modified release component having a modified release matrix material are prepared according to the formulations shown in Table 5(a) and (b).

TABLE 7 (a)
100 mg of IR component is encapsulated with 100 mg of modified
release (MR) component to give a 20 mg dosage strength product

| | % (w/w) |
|---|---|
| IR component | |
| Vitamin K2 | 10 |
| Microcrytalline cellulose | 40 |
| Lactose | 45 |
| Povidone | 5 |
| MR component | |
| Vitamin K2 | 10 |
| Microcrytalline cellulose | 40 |
| Eudragit ® RS | 45 |
| Povidone | 5 |

TABLE 7 (b)
50 mg of IR component is encapsulated with 50 mg of modified
release (MR) component to give a 20 mg dosage strength product.

| | % (w/w) |
|---|---|
| IR component | |
| Vitamin K2 | 20 |
| Microcrystalline cellulose | 50 |
| Lactose | 28 |
| Povidone | 2 |
| MR component | |
| Vitamin K2 | 20 |
| Microcrytalline cellulose | 50 |
| Eudragit ® S | 28 |
| Povidone | 2 |

### Example 3

The purpose of this example was to prepare nanoparticulate vitamin K2 compositions using various combinations of surface stabilizers and milling times.

An aqueous dispersion of vitamin K2 (menatetrenone) combined with one or more surface stabilizers, at the concentrations shown in Table 8, below, was milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). All compositions were milled for 60 min. at a speed of 2500.

TABLE 8
Vitamin K2 Formulations

| | | | Deionized |
|---|---|---|---|
| | Menatetrenone | | Water |
| Sample | Concentration | Surface Stabilizer(s) | (w/w) |
| 1 | 5% (w/w) | 2% (w/w) Pharmacoat ® 603 | 93% |
| | | (hydroxypropylmethylcellulose) | |
| 2 | 5% (w/w) | 1.25% (w/w) HPC-SL | 93.7% |
| | | (hydroxypropylcellulose) | |
| | | 0.05% (w/w) docusate sodium | |
| 3 | 5% (w/w) | 1.5% (w/w) Pluronic ® (Lutrol) | 93.5% |
| | | F-108 (Poloxamer 338) | |
| 4 | 5% (w/w) | 1.25% (w/w) Plasdone K-17 | 93.7% |
| | | (Polyvinylpyrrolidone K-17) | |
| | | 0.05% (w/w) Benzalkonium | |
| | | chloride | |
| 5 | 5% (w/w) | 1.25% (w/w) Tyloxapol | 93.75% |
| 6 | 5% (w/w) | 1.25% (w/w) Lutrol (Pluronic) | 93.7% |
| | | F68 (Poloxamer 188) | |
| | | 0.05% (w/w) Docusate Sodium | |
| 7 | 5% (w/w) | 1.25% (w/w) Plasdone K29/32 | 93.7% |
| | | (Polyvinylpyrrolidone K29/32), | |
| | | 0.05% (w/w) Sodium Lauryl | |
| | | Sulfate | |
| 8 | 5% (w/w) | 1.25% (w/w) Plasdone C-15 | 93.7% |
| | | (Polyvinylpyrrolidone C-15) | |
| | | 0.05% (w/w) Deoxycholic acid | |
| | | sodium salt | |
| | | (Sodium Deoxycholate) | |
| 9 | 5% (w/w) | 1.5% (w/w) Tween 80 | 93.5% |
| | | (Polysorbate 80) | |
| 10 | 5% (w/w) | 0.1% w/w Docusate Sodium | 94.9% |
| 11 | 5% (w/w) | 1.25% (w/w) Plasdone S-630 | 93.7% |
| | | (Polyvinylpyrrolidone) | |
| | | 0.05% (w/w) Sodium Lauryl | |
| | | Sulfate | |
| 12 | 5% (w/w) | 1.0% (w/w) Lutrol (Pluronic) | 93.0% |
| | | F-108 (Poloxamer 338) | |
| | | 1.0% (w/w) Tween 80 | |
| | | (Polysorbate 80) | |

The milled compositions were harvested using a 21 gauge syringe and analyzed via microscopy. Microscopy was done using a Lecia DM5000B and Lecia CTR 5000 light source (Laboratory Instruments and Supplies Ltd., Ashbourne Co., Meath, Ireland). The microscopy observations for each formulation are shown below in Table 9.

TABLE 9

| Formulation | Microscopy Observations |
|---|---|
| 1 | Microscopy showed clearly the presence of discrete |
| | vitamin K2 nanoparticles which were observed to exhibit |
| | brownian motion. There was no sign of flocculation |
| | present when imaging the aliquot of nanoparticulate |
| | vitamin K2. |
| 2 | The sample displayed discrete vitamin K2 nanoparticles |
| | which were well dispersed. Brownian motion was clearly |
| | evident. There was no evidence of flocculation or crystal |
| | growth present. |
| 3 | Microscopy showed the presence of discrete vitamin K2 |
| | nanoparticles which exhibited brownian motion. There |
| | was no signs of flocculation observed when imaging the |
| | aliquot of nanoparticulate vitamin K2. There were small |
| | numbers of unmilled/partially milled vitamin K2 crystals |
| | present from microscopy observations. |
| 4 | This sample appeared to be well dispersed with vitamin |
| | K2 nanoparticles present. Some larger vitamin K2 |
| | particles were observed throughout the sample, however |
| | these did not appear to be larger than 2000 nm. Brownian |
| | motion was also clearly evident. |
| 5 | The sample from microscopy showed to be reasonably |
| | well dispersed with some small signs of unmilled vitamin |
| | K2 visible. No signs of flocculation present. Brownian |
| | motion was also visible. |
| 6 | The sample from the microscopy showed to be |
| | reasonably dispersed with signs of vitamin K2 |
| | nanoparticulates visible as well as some unmilled vitamin |
| | K2 present. Some smaller localized flocculation was |
| | present also. |
| 7 | Discrete vitamin K2 nanoparticulates were present which |
| | displayed brownian motion. Some larger vitamin K2 |
| | particles were also observed which may be partially |
| | milled vitamin K2. |
| 8 | Microscopy showed the sample to be highly flocculated |
| | which was consistent with the particle size data. |
| | Brownian motion and vitamin K2 nanoparticulates were |
| | also observed. |
| 9 | Discrete vitamin K2 nanoparticulates were present which |
| | displayed brownian motion. There was no evidence of |
| | flocculation or crystal growth present in the sample. |
| 10 | Discrete vitamin K2 nanoparticles were present in the |
| | micrograph. Brownian motion was also clearly evident. |
| | There was evidence of crystal growth or flocculation. It |
| | was noted however that fewer vitamin K2 nanoparticles |
| | were visible than would be expected. |
| 11 | The sample from microscopy showed to be well dispersed |
| | with some very small amounts of immilled vitamin K2 |
| | present. No signs of flocculation present. Brownian |
| | motion was also visible. |
| 12 | Microscopy showed the sample to be well dispersed with |
| | vitamin K2 nanoparticulates clearly visible which |
| | exhibited brownian motion. There was no flocculation |
| | observed during analysis. |

The particle size of the milled vitamin K2 particles was measured, in Milli Q Water, using a Horiba LA-910 Particle Sizer (Particular Sciences, Hatton Derbyshire, England). Vitamin K2 particle size was measured initially and then again following 60 seconds sonication. The results are shown below in Table 10.

TABLE 10

| | Mean | D50 | D90 | D95 | | |
|---|---|---|---|---|---|---|
| Sample | (nm) | (nm) | (nm) | (nm) | Sonication? | Comments |
| 1 | 164 | 177 | 242 | 266 | N | The resulting slurry did not appear to |
| | 162 | 175 | 240 | 263 | Y | be viscous in nature. The color of the |
| | | | | | | nanoparticulate vitamin K2 dispersion |
| | | | | | | was yellow, similar to that of the |
| | | | | | | starting active agent. The |
| | | | | | | nanoparticulate vitamin K2 dispersion |
| | | | | | | did not appear to solubilize when |
| | | | | | | added to Milli Q water during particle |
| | | | | | | size analysis. |
| 2 | 173 | 167 | 226 | 252 | N | The nanoparticulate vitamin K2 |
| | 171 | 166 | 224 | 249 | Y | dispersion was yellow in color and |
| | | | | | | appeared to have a low viscosity which |
| | | | | | | harvested easily. |
| 3 | 224 | 213 | 293 | 333 | N | The resulting slurry did not appear to |
| | 220 | 210 | 289 | 329 | N | be viscous in nature. The |
| | 220 | 209 | 288 | 326 | Y | nanoparticulate vitamin K2 dispersion |
| | | | | | | was observed to be yellow in color, |
| | | | | | | similar to that of the starting API. The |
| | | | | | | nanoparticulate vitamin K2 dispersion |
| | | | | | | did not appear to solubilise when |
| | | | | | | added to Milli Q water during particle |
| | | | | | | size analysis. A repeat analysis of the |
| | | | | | | initial run (no sonication) was |
| | | | | | | performed as the Lamp % T value was |
| | | | | | | outside of specified range. |
| 4 | 305 | 276 | 431 | 526 | N | The milled nanoparticulate vitamin K2 |
| | 291 | 264 | 403 | 490 | Y | dispersion was a yellow, |
| | | | | | | homogeneous, low viscosity liquid |
| | | | | | | dispersion with no signs of gelation. |
| 5 | 136 | 134 | 185 | 202 | N | The milled nanoparticulate vitamin K2 |
| | 138 | 134 | 185 | 202 | Y | dispersion was yellow in color with |
| | | | | | | low viscosity. There was no signs of |
| | | | | | | gelling or aggregation at this point |
| 6 | 235 | 221 | 310 | 357 | N | The milled nanoparticulate vitamin K2 |
| | 230 | 218 | 301 | 342 | Y | dispersion was yellow in appearance |
| | | | | | | with low viscosity. There was no signs |
| | | | | | | of gelling or aggregation |
| 7 | 260 | 216 | 322 | 405 | N | The nanoparticulate vitamin K2 |
| | 255 | 217 | 321 | 397 | Y | dispersion was yellow in color and |
| | | | | | | appeared to have a low viscosity. |
| 8 | 29150 | 20476 | 71453 | 88370 | N | The nanoparticulate vitamin K2 |
| | 1326 | 417 | 3807 | 5491 | Y | dispersion was observed to be yellow |
| | | | | | | in color and agglomerated. It appeared |
| | | | | | | to have a high viscosity. |
| 9 | 548 | 165 | 236 | 284 | N | The nanoparticulate vitamin K2 |
| | 166 | 161 | 219 | 241 | Y | dispersion appeared yellow in color |
| | 166 | 161 | 221 | 247 | N | with a low viscosity. Two repeat |
| | 163 | 159 | 216 | 238 | Y | analyses were performed on the |
| | 364 | 163 | 227 | 261 | N | harvested nanoparticulate vitamin K2 |
| | 162 | 158 | 215 | 236 | Y | dispersion due to a small secondary |
| | | | | | | distribution being present in the data. |
| | | | | | | The secondary distribution was |
| | | | | | | observed in Milling 1 sample and in |
| | | | | | | Repeat 2. It did not appear in the |
| | | | | | | Repeat 1 sample suggesting it not to be |
| | | | | | | a reproducible result. All data met the |
| | | | | | | acceptance criteria and was therefore |
| | | | | | | not further investigated. |
| 10 | 222 | 203 | 317 | 378 | N | The nanoparticulate vitamin K2 |
| | 206 | 194 | 293 | 338 | Y | dispersion was yellow in color and |
| | | | | | | appeared to have a low viscosity. Some |
| | | | | | | material had adhered to the agitator and |
| | | | | | | chamber and had formed into a paste- |
| | | | | | | like substance. |
| 11 | 174 | 168 | 233 | 257 | N | The milled nanoparticulate vitamin K2 |
| | 176 | 169 | 235 | 259 | Y | dispersion was yellow in color and |
| | | | | | | homogeneous with low viscosity and |
| | | | | | | no signs of gelling or aggregation |
| 12 | 174 | 167 | 234 | 260 | N | The nanoparticulate vitamin K2 |
| | 171 | 165 | 226 | 253 | Y | dispersion was observed to have a low |
| | 175 | 167 | 234 | 259 | N | viscosity with no agglomeration. The |
| | 171 | 165 | 226 | 252 | Y | sample appeared homogeneous. |
| | | | | | | Repeat analysis was performed, as |
| | | | | | | initial particle size data was collected |
| | | | | | | at a Lamp % T value outside of |
| | | | | | | specified Lamp % T range. |

Particle sizes that vary significantly following sonication, such as that observed for Sample (Formulation) 8 in Table 10, are undesirable, as it is indicative of the presence of vitamin K2 aggregates. Such aggregates result in compositions having highly variable particle sizes. Such highly variable particle sizes can result in variable absorption between dosages of a drug, and therefore are undesirable.

The data demonstrate the successful preparation of nanoparticulate vitamin K2 formulations utilizing various surface stabilizers, including various combination of surface stabilizers.

It will be apparent to those skilled in the art that various modifications and variations can be made in the methods and compositions of the present inventions without departing from the spirit or scope of the invention. Thus, it is intended that the present invention cover the modification and variations of the invention provided they come within the scope of the appended claims and their equivalents.

What is claimed is:

1. A stable nanoparticulate vitamin K2 composition comprising:
(a) particles of a vitamin K2 having an effective average particle size of less than about 2000 nm; and
(b) at least one surface stabilizer.

2. The composition of claim 1, wherein the nanoparticulate vitamin K2 particles have improved bioavailability as compared to conventional vitamin K2 tablets.

3. The composition of claim 1, wherein the nanoparticulate vitamin K2 particle is selected from the group consisting of a crystalline phase, an amorphous phase, a semi-crystalline phase, a semi amorphous phase, and mixtures thereof.

4. The composition of claim 3, wherein the effective average particle size of the nanoparticulate vitamin K2 particles is selected from the group consisting of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 100 nm, less than about 75 nm, and less than about 50 nm.

5. The composition of claim 1, wherein the composition is formulated:
(a) into a dosage form selected from the group consisting of tablets, capsules, sachets, solutions, gels, aerosols, ointments, creams, dispersions, and mixtures thereof;
(b) into a dosage form selected from the group consisting of controlled release formulations, fast melt formulations, lyophilized formulations, delayed release formulations, extended release formulations, pulsatile release formulations, and mixed immediate release and controlled release formulations; or
(c) a combination of (a) and (b).

6. The composition of claim 1, wherein the composition further comprises one or more pharmaceutically acceptable excipients, carriers, or a combination thereof.

7. The composition of claim 1, wherein:
(a) vitamin K2 is present in an amount consisting of from about 99.5% to about 0.001%, from about 95% to about 0.1%, and from about 90% to about 0.5%, by weight, based on the total combined weight of vitamin K2 and at least one surface stabilizer, not including other excipients;
(b) at least one surface stabilizer is present in an amount of from about 0.5% to about 99.999% by weight, from about 5.0% to about 99.9% by weight, and from about 10% to about 99.5% by weight, based on the total combined dry weight of vitamin K2 and at least one surface stabilizer, not including other excipients; or
(c) a combination of (a) and (b).

8. The composition of claim 1, wherein the surface stabilizer is selected from the group consisting of a non-ionic surface stabilizer, an anionic surface stabilizer, a cationic surface stabilizer, a zwitterionic surface stabilizer, and an ionic surface stabilizer.

9. The composition of claim 1, wherein the surface stabilizer is selected from the group consisting of cetyl pyridinium chloride, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hypromellose, carboxymethylcellulose sodium, methylcellulose, hydroxyethylcellulose, hypromellose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctylsulfosuccinate, dialkylesters of sodium sulfosuccinic acid, sodium lauryl sulfate, allyl aryl polyether sulfonates, mixtures of sucrose stearate and sucrose distearate, p-isononylphenoxypoly-(glycidol), decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; lysozyme, PEG-phospholipid, PEG-cholesterol, PEG-cholesterol derivative, PEG-vitamin A, PEG-vitamin E, lysozyme, random copolymers of vinyl acetate and vinyl pyrrolidone, a cationic polymer, a cationic biopolymer, a cationic polysaccharide, a cationic cellulosic, a cationic alginate, a cationic nonpolymeric compound, a cationic phospholipid, cationic lipids, polymethylmethacrylate trimethylammonium bromide, sulfonium compounds, polyvinylpyrrolidone-2-dimethylaminoethyl methacrylate dimethyl sulfate, hexadecyltrimethyl ammonium bromide, phosphonium compounds, quarternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅dimethyl hydroxyethyl ammonium chloride, C₁₂₋₁₅-dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl (ethenoxy)₄ ammonium chloride, lauryl dimethyl (ethenoxy)₄ ammonium bromide, N-alkyl (C₁₂₋₁₈)dimethylbenzyl ammonium chloride, N-alkyl (C₁₄₋₁₈)dimethylbenzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, C₁₅ trimethyl ammonium bromides, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, POLYQUAT 10™, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, MIRAPOL™, ALKAQUATT™, alkyl pyridinium salts; amines, amine salts, amine oxides, imide azolinium salts, protonated quaternary acrylamides, methylated quaternary polymers, and cationic guar.

10. The composition of claim 1, additionally comprising one or more active agents useful in the prevention or treatment of osteoporosis.

11. The composition of claim 10, wherein the one or more active agents is selected from the group consisting of risedronic acid, estrogens, calcitonins, and bisphoshonates.

12. The composition of claim 1, wherein the composition does not produce significantly different absorption levels when administered under fed as compared to fasting conditions.

13. The composition of claim 1, wherein the pharmacokinetic profile of the composition is not significantly affected by the fed or fasted state of a subject ingesting said composition.

14. The composition of claim 1, wherein administration of the composition to a subject in a fasted state is bioequivalent to administration of the composition to a subject in a fed state.

15. A method of preparing a nanoparticulate vitamin K2 composition comprising contacting particles of a vitamin K2 with at least one surface stabilizer for a time and under conditions sufficient to provide a nanoparticulate vitamin K2 composition having an effective average particle size of less than about 2000 nm.

16. The method of claim 28, wherein the contacting comprises grinding, wet grinding, homogenization, freezing, template emulsion, precipitation, or a combination thereof.

17. The method of claim 15, wherein the effective average particle size of the nanoparticulate vitamin K2 particles is selected from the group consisting of less than about 1900 nm, less than about 1800 nm, less than about 1700 nm, less than about 1600 nm, less than about 1500 nm, less than about 1000 nm, less than about 1400 nm, less than about 1300 nm, less than about 1200 nm, less than about 1100 nm, less than about 900 nm, less than about 800 nm, less than about 700 nm, less than about 600 nm, less than about 500 nm, less than about 400 nm, less than about 300 nm, less than about 250 nm, less than about 200 nm, less than about 100 nm, less than about 75 nm, and less than about 50 nm.

18. A method of preventing and/or treating osteoporosis comprising administering a nanoparticulate vitamin K2 composition comprising:
(a) particles of a vitamin K2 having an effective average particle size of less than about 2000 nm; and
(b) at least one surface stabilizer.

19. The method of claim 18, wherein the effective average particle size of the nanoparticulate vitamin K2 particles is selected from the group consisting of less than about 1900 nm, less than about 1800 nm, less than about 1700 nm, less than about 1600 nm, less than about 1500 nm, less than about 1000 nm, less than about 1400 nm, less than about 1300 nm, less than about 1200 nm, less than about 1100 nm, less than about 900 nm, less than about 800 nm, less than about 700 nm, less than about 600 nm, less than about 500 nm, less than about 400 nm, less than about 300 nm, less than about 250 nm, less than about 200 nm, less than about 100 nm, less than about 75 nm, and less than about 50 nm.

20. A controlled-release composition comprising a population of vitamin K2-containing particles wherein the particles comprise a modified-release coating or, alternatively or additionally, a modified-release matrix material, such that, following oral delivery of the composition to a subject, the composition delivers vitamin K2 in a pulsatile or continuous manner.

21. A composition according to claim 20 wherein said population is an erodable formulation.

22. A composition according to claim 20 wherein said particles comprise a modified-release coating.

23. A composition according to claim 20 wherein particles comprise a modified-release matrix material.

24. A composition according to claim 22 or 23 wherein said particles are combined in a formulation that releases said vitamin K2 by erosion to the surrounding environment.

25. A composition according to claim 20 which comprises also an enhancer.

26. A composition according to claim 20 wherein said particles are contained in a hard gelatin or soft gelatin capsule.

27. A composition according to claim 20 wherein said particles are in the form of mini-tablets.

28. A composition according to claim 20 in the form of a tablet wherein the particles are compressed to form a layer of said tablet.

29. A composition according to claim 20 wherein said particles are provided in a rapidly dissolving dosage form.

30. A composition according to claim 20 in the form of a fast-melt tablet.

31. A composition according to claim 20 wherein said particles comprise a pH-dependent polymer coating which is effective in releasing a pulse of vitamin K2 after a time delay of six to twelve hours.

32. The composition according to claim 31 wherein said polymer coating comprises methacrylate copolymers.

33. The composition according to claim 31 wherein said polymer comprises a mixture of methacrylate and ammonio methacrylate copolymers in a ratio sufficient to achieve a pulse of vitamin K2 following a time delay.

34. The composition according to claim 33 wherein said ratio is approximately 1:1.

35. The composition according to claim 20 wherein said Vitamin K2 is in nanoparticulate form.

36. The composition according to claim 35 wherein said composition does not produce significantly different absorption levels when administered under fed conditions as compared to fasting conditions.

37. The composition according to claim 35 wherein the pharmacokinetic profile of said composition is not significantly affected by the fed or fasted state of a subject ingesting said composition.

38. The composition according to claim 35 wherein the administration of said composition to a subject in a fasted state is bioequivalent to the administration of said composition to a subject in a fed state.

39. A controlled-release composition comprising: (A) a first component comprising a first population of a vitamin K2; and (B) a subsequent component comprising a subsequent population of vitamin K2; said composition being capable of delivering vitamin K2 in a pulsatile or continuous manner.

40. A composition according to claim 39 wherein said first component allows for the immediate-release of vitamin K2.

41. A composition according to claim 39 wherein said first component is a time-delayed immediate release component.

42. A composition according to claim 39 wherein said subsequent component comprises a modified-release coating or, alternatively or additionally, a modified-release matrix material.

43. A composition according to claim 39 wherein said subsequent component is a time-delayed immediate release component.

44. A composition according to claim 39 wherein said first component is a time-delayed immediate release component.

45. A composition according to claim 39 that delivers vitamin K2 in a pulsatile manner.

46. A composition according to claim 39 that delivers vitamin K2 in a continuous manner.

47. A composition according to claim 39 wherein the vitamin K2 in at least one of said components is nanoparticulate vitamin K2.

48. A method for the prevention and/or treatment of osteoporosis comprising administering a therapeutically effective amount of a composition according to claim 20.

49. A method for the prevention and/or treatment of osteoporosis comprising administering a therapeutically effective amount of a composition according to claim 39.

50. A stable nanoparticulate composition comprising:
(a) particles comprising vitamin K2 having an effective average particle size of less than about 2000 nm; and
(b) at least one surface stabilizer,
wherein upon administration to a mammal the composition produces therapeutic results at a dosage which is less than that of a non-nanoparticulate dosage form of the same vitamin K2.

51. A composition comprising a vitamin K2, wherein the composition has:
(a) a Cₘₐₓ for the vitamin K2 when assayed in the plasma of a mammalian subject following administration that is greater than the Cₘₐₓ for a non-nanoparticulate formulation of the same vitamin K2, administered at the same dosage;
(b) an AUC for the vitamin K2 when assayed in the plasma of a mammalian subject following administration that is greater than the AUC for a non-nanoparticulate formulation of the same vitamin K2, administered at the same dosage;
(c) a Tₘₐₓ for the vitamin K2 when assayed in the plasma of a mammalian subject following administration that is less than the Tₘₐₓ for a non-nanoparticulate formulation of the same vitamin K2, administered at the same dosage; or
(d) any combination of (a), (b), and (c).
